(19) United States
(12) Patent Application Publication
(10) Pub. No.: US 2025/0006297 A1
(43) Pub. Date: Jan. 2, 2025

(54) PREDICTING EMBRYO PLOIDY STATUS USING TIME-LAPSE IMAGES

(71) Applicant: CORNELL UNIVERSITY, Ithaca, NY (US)

(72) Inventors: Iman HAJIRASOULIHA, New York, NY (US); Nikica ZANINOVIC, New York, NY (US); Josue BARNES, New York, NY (US); Zev ROSENWAKS, New York, NY (US); Olivier ELEMENTO, New York, NY (US); Jonas MALMSTEN, Gibsonton, FL (US); Suraj RAJENDRAN, New York, NY (US)

(73) Assignee: CORNELL UNIVERSITY, Ithaca, NY (US)

(21) Appl. No.: 18/437,062

(22) Filed: Feb. 8, 2024

Related U.S. Application Data
(60) Provisional application No. 63/484,177, filed on Feb. 9, 2023.

(51) Int. Cl. G16B 20/10; G16H 30/40; G16H 50/30; G16H 50/70

(52) U.S. Cl. CPC G16B 20/10; G16H 30/40; G16H 50/30; G16H 50/70

(57) ABSTRACT

The present disclosure encompasses systems and methods for predicting embryo ploidy. Specific embodiments encompass methods of non-invasively predicting ploidy status of an embryo, by receiving a dataset with video including a plurality of image frames of the embryo, analyzing the plurality of image frames by one or more machine and/or deep learning model via one or more classification task applied to the dataset; and generating an output prediction of the ploidy status of the embryo. Particular methods relate to methods wherein the dataset additionally includes one or more clinical and/or morphological features for the embryo, such as maternal age at the time of oocyte retrieval. Embodiments also relate to predicting embryo viability and/or improving embryo selection, such as during in vitro fertilization, and uses thereof.

### CROSS REFERENCE TO RELATED APPLICATIONS

The present application claims the benefit of priority under 35 U.S.C. § 119(e) to U.S. Provisional Application No. 63/484,177, PREDICTING EMBRYO PLOIDY STATUS USING TIME-LAPSE AND SINGLE IMAGES, filed on Feb. 9, 2023, which is currently co-pending herewith and which is incorporated by reference in its entirety.

### STATEMENT REGARDING FEDERALLY-SPONSORED RESEARCH

This invention was made with government support under Grant Nos. R35 GM138152-01, 1T32 GM083937, and TL1-TR-002386 awarded by the National Institutes of Health. The government has certain rights in the invention.

### FIELD

The present disclosure relates generally to the field of assisted reproduction, and particularly relates to systems, software, and methods for evaluating embryos by, for example, predicting embryo ploidy status.

### BACKGROUND

A challenge in the field of in vitro fertilization (IVF) is the selection of the most viable embryos for transfer. Current methods of embryo selection include morphological quality assessment and morphokinetic analysis; however, both of these methods suffer from intra- and inter-observer bias and variability due to the unstandardized methods, as observed in morphological assessment and morphokinetic annotation. A third method applied to embryo selection involves pre-implantation genetic testing for aneuploid (PGT-A); this method also has notable limitations, including the invasive nature of trophectoderm biopsies, which leads to ethical issues, and high cost. As such, there is a need in the industry to provide automated, non-invasive tools for evaluating and selecting embryos for transfer during IVF.

Several recent studies have sought to alleviate the limitation of morphological assessment by utilizing deep learning to predict embryo quality. However, fewer studies, have sought to use deep learning to predict embryo ploidy as a standardized method of embryo selection.

Differences in aneuploid and euploid embryos that allow for model-based classification are reflected in morphology, morphokinetics, and associated clinical information. As such, there is a need in the industry to provide automated, non-invasive tools for evaluating and selecting embryos for transfer during IVF via model-based classification.

To meet this need, a non-invasive and automated method of embryo evaluation was developed that uses deep learning with image and video classification to predict embryo ploidy status (also referred herein as the model-derived video-based blastocyst score and ploidy classification model, the model-derived video-based blastocyst score and ploidy classification/prediction model, the model-derived video-based blastocyst score and ploidy prediction model, or as the MDBS-Ploidy or MDBS ploidy model).

Given that chromosomal abnormalities impact embryo development, the differences in aneuploid and euploid embryos that allow for model-based classification are reflected in morphology, morphokinetics, and associated clinical information. The various models discussed herein demonstrate an ability to predict blastocyst ploidy in an non-invasive manner as an improvement to traditional and inferior methods.

### SUMMARY

Embodiments of the disclosure relate to non-invasive methods of predicting ploidy status of an embryo, the methods including: receiving a dataset including video including a plurality of image frames of the embryo; analyzing the dataset by one or more machine and/or deep learning model via one or more classification task applied to the dataset; and generating an output prediction of the ploidy status of the embryo.

In some embodiments, the prediction of the ploidy status of the embryo comprises a probability. In some embodiments, the probability comprises a probability of the embryo being euploid.

In some embodiments, the classification task can be a binary classification task. In some embodiments, the binary classification task can provide a probability for the embryo of being euploid vs. aneuploid; or euploid vs complex aneuploid. In some embodiments, the binary classification task can provide a probability for the embryo of being euploid vs. aneuploid. In some embodiments, the binary classification task can provide a probability for the embryo of being euploid vs. complex aneuploid.

In some embodiments, the methods can further include acquiring the plurality of image frames. In some embodiments, the plurality of image frames can be acquired via time-lapse microscopy. In some embodiments, the plurality of image frames can be captured at Day 5 of embryo development. In some embodiments, each image of the plurality of image frames can be captured from 96-112 hours post insemination (hpi). In some embodiments, the plurality of image frames can include one, two, three, four, five, or more image frames captured per hour for two or more consecutive or non-consecutive hours during Day 5 of embryo development. In some embodiments, the plurality of image frames can include one, two, three, four, five, or more image frames captured per hour for each hour of Day 5 of embryo development. In some embodiments, the plurality of image frames can include two, three, four, five, or more image frames captured and analyzed per embryo.

In some embodiments, the model can further generate an output including one or more clinical and/or morphological feature scores for the embryo. In some embodiments, the one or more clinical and/or morphological feature scores for the embryo can include blastocyst score (BS), expansion score (ES), inner-cell mass (ICM) score, and/or trophectoderm (TE) score. In some embodiments, the one or more clinical and/or morphological feature scores for the embryo can include blastocyst score (BS). In some embodiments, the dataset can further include one or more clinical and/or morphological features for the embryo. In some embodiments, the one or more clinical features for the embryo can include maternal age at the time of oocyte retrieval. In some embodiments, the one or more clinical and/or morphological features for the embryo can include one or more morphokinetic parameters/annotations, one or more blastocyst morphological assessments, and/or preimplantation genetic testing for aneuploidy (PGT-A). In some embodiments, the blastocyst morphological assessments can include blastocyst grade (BG), blastocyst score (BS), time to blastocyst (tB), and/or artificial intelligence-driven predicted blastocyst score (AIBS). In some embodiments, the BS score determination can include converting inner cell mass (ICM), trophectoderm (TE), and/or expansion grades into numerical values, and additionally can include an input based on day of blastocyst formation. In some embodiments, blastocyst score can include a numerical value based on ICM, TE, and expansion grade, and/or a score based on day of blastocyst formation. In some embodiments, the morphokinetic parameters can include time of pro-nuclear fading (tPnF), time to 2 cells (t2), time to 3 cells (t3), time to 4 cells (t4), time to 5 cells (t5), time to 6 cells (t6), time to 7 cells (t7), time to 8 cells (t8), time to 9 cells (t9), time of morula (tM), and/or time of the start of blastulation (tSB). In some embodiments, analyzing morphokinetic parameters can include assigning blastocyst grade (BG) using a grading system. In some embodiments, the grading system can include assessments of inner cell mass (ICM), trophectoderm (TE), and/or expansion. In some embodiments, maternal age and/or blastocyst score (BS) can be weighted more heavily than other clinical features based on one or more classification task. In some embodiments, the clinical and/or morphological features can be weighted in order of maternal age at the time of oocyte retrieval, blastocyst grade and/or blastocycst score, and/or morphokinetic parameters. In some embodiments, blastocyst score can be correlated positively, and/or maternal age can be correlated negatively with embryo ploidy status. In some embodiments, the maternal age is 37 or younger and wherein the embryo has a higher probability of being euploid.

In some embodiments, the methods can further include pre-processing the dataset prior to analysis. In some embodiments, pre-processing the dataset can include removing faulty image frames and/or imputing values for any missing image frames via median imputation. In some embodiments, a faulty image can include an image that cannot be processed and/or analyzed.

In some embodiments, the output prediction can be determined based on machine and/or deep learning and regression analysis. In some embodiments, the analysis can include regression analysis. In some embodiments, the regression analysis can include a LASSO regression and/or logistic regression applied to the plurality of image frames and/or one or more clinical and/or morphological features. In some embodiments, the analysis can include determination of an artificial intelligence-driven predicted blastocyst score (AIBS) for the embryo. In some embodiments, the image frames and/or clinical features can be combined and analyzed by machine and/or deep learning in two fully-connected layers.

In some embodiments, the analysis can output a predicted embryo ploidy in a binary classification task. In some embodiments, the machine learning can include a convolutional neural network (CNN). In some embodiments, the machine learning can include a Bidirectional Long Short-Term Memory (BiLSTM) network.

Some embodiments of the methods can further include: training the one or more machine learning model using training data, wherein the training data includes a plurality of probabilities, and/or model- or embryologist-derived or provided clinical features for a plurality of subjects and a plurality of embryo ploidy statuses for the plurality of subjects.

Some embodiments of the methods can further include predicting embryo viability based on the embryo ploidy status, wherein an embryo having a stronger probability of being euploid has a higher probability of being viable. In some embodiments, the methods can be used for improving embryo selection for implantation during in vitro fertilization. In some embodiments, the methods can be used for selecting and/or prioritizing an embryo for preimplantation genetic testing for aneuploidy (PGT-A) biopsy and/or implantation during in vitro fertilization. In some embodiments, the methods can be used in combination with traditional methods of embryo selection and prioritization for implantation and/or recommendation for PGT-A during in vitro fertilization.

Further embodiments of the disclosure include methods of improving an outcome in a subject undergoing in vitro fertilization, including the aforementioned methods, wherein an embryo predicted to be euploid is selected for embryo transfer during in vitro fertilization, and/or wherein an embryo predicted to be aneuploid is not selected for embryo transfer during in vitro fertilization, thus improving an outcome by selecting for an embryo.

In some embodiments, one or more aspects of the methods of the disclosure can be automated.

Further embodiments of the disclosure relate to systems including: one or more data processors; and a non-transitory computer readable storage medium containing instructions which, when executed on the one or more data processors, cause the one or more data processors to perform part or all of the any of the aforementioned methods.

Further embodiments of the disclosure relate to computer-program products tangibly embodied in a non-transitory machine-readable storage medium, including instructions configured to cause one or more data processors to perform part or all of any of the aforementioned methods.

Further embodiments of the disclosure relate to user interfaces for predicting ploidy status of an embryo, the user interface including: a web-based platform for uploading and analyzing a dataset, wherein the dataset includes a video comprising a plurality of image frames of the embryo; analysis software integrated with the web-based platform to analyze the dataset by one or more machine and/or deep learning model via one or more classification task applied to the dataset; and an output generation which provides a prediction of ploidy status of the embryo.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. An example computer system, upon which embodiments, or portions of the embodiments, may be implemented, in accordance with various embodiments.

FIG. 2. Overview of the workflow and methodology used for ploidy prediction and classification via compare analysis based on single static images as input with analysis based on video as input.

FIGS. 3A-3D. Model performances of classification models across time periods and prediction tasks. FIG. 3A depicts average AUC with standard error bars across different days and different image classification tasks. FIG. 3B depicts macro ROC plots for each image classification prediction task. FIG. 3C depicts average AUC with standard error bars across different days and different video classification tasks. FIG. 3D depicts macro ROC plots for each video classification prediction task.

FIGS. 4A-4B. Model performances of classification models without maternal age across time periods on Embryoscope and Embryoscope+ datasets. FIG. 4A depicts average AUC with standard error bars across different days and different video classification tasks. FIG. 4B depicts average AUC with standard error bars across different days and different image classification tasks.

FIGS. 5A-5B. Intercluster differences between embryos clustered by ploidy status. Intercluster differences, measured as distance between cluster centroids, is shown for all Embryoscope embryos. FIG. 5A depicts intercluster difference at each timepoint (i.e. hours post insemination) for euploid (EUP) vs aneuploid (ANU). FIG. 5B depicts intercluster difference at each timepoint (i.e. hours post insemination) for EUP vs. complex aneuploidy (CxA). Black lines are gaussian smoothed curves that show the general trend of intercluster differences.

FIG. 6. Model performances of Day 5 image and video classification models.

FIGS. 7A-7B. Performance of models with different features across all settings. Average AUC with standard errors are shown for all prediction tasks on both Embryoscope testset and Embryoscope+datasets. FIG. 7A depicts models with only one feature type (image, video, age, blastocyst score, or time to blastulation). FIG. 7B depicts performance of all models with age as a feature.

FIG. 8. Overview of development of the model-derived video-based blastocyst score and ploidy classification model. Features can be extracted from time-lapse image frames as shown in FIG. 2, steps 1-5. These features can be fed into a multitask BiLSTM model which is trained to predict blastocyst score as well as other embryologist-annotated morphological scores, and predicted blastocyst scores can then be inputted into a logistic regression model to perform ploidy classification.

FIGS. 9A-9B. Correlations between predicted and actual BS scores from the model-derived video-based blastocyst score and ploidy classification model. Pearson correlation scores are shown in panel title (PS=*). FIG. 9A depicts correlations within the training set. FIG. 9B shows correlations within the Embyoscope test set.

FIGS. 10A-10F. Correlations between predicted and actual multitask scores from the model-derived video-based blastocyst score and ploidy classification model. Correlation plots for other predicted scores in the model-derived video-based blastocyst score and ploidy classification model (not used in downstream processes) are shown; Pearson correlations are shown in panel title (PS=*). FIG. 10A, FIG. 10C, and FIG. 10E depict correlations within the training set. FIG. 10B, FIG. 10D, and FIG. 10F depict correlations within the Embyoscope test set.

FIGS. 11A-11B. Comparison of the model-derived video-based blastocyst score and ploidy classification model with other models. FIG. 11A depicts performances of models without maternal age.

FIG. 11B depicts performances of models with maternal age

FIG. 12. Performances of Day 5 video and the model-derived video-based blastocyst score and ploidy classification model on Spain dataset.

### DETAILED DESCRIPTION

### I. Overview

This specification describes various exemplary embodiments of systems, software and methods for evaluating embryos by, for example, predicting embryo ploidy status. The disclosure, however, is not limited to these exemplary embodiments and applications or to the manner in which the exemplary embodiments and applications operate or are described herein.

With the advent of artificial intelligence, deep learning models have been developed to supplement embryologist workflows. Current models, however, do not effectively utilize time-lapse imaging nor provide information on what time frames are of most importance.

As described herein, the present disclosure relates to the design and utilization of various embryo ploidy status prediction models and compares their performances across different time points of embryo development. Model performance is used to determine what periods of embryo development provide information for detecting ploidy status. Model performances is also compared between image and video classification to determine if added temporal information from sequences of time-lapse images provides any advantage.

Various embodiments of the disclosure relate to an effective model developed for ploidy prediction, as described herein. In particular, a non-invasive method of predicting ploidy status of an embryo has been developed, based on analyzing a dataset including video image frames of the embryo, by one or more machine and/or deep learning model via one or more classification task applied to the dataset; andgenerating an output prediction of the ploidy status of the embryo. The machine and/or deep learning model can be trained to provice classification or a prediction of the probability of the embryo being euploid (EUP) vs aneuploid (ANU), EUP vs simple aneuploid (SA), EUP vs. complex aneuploid (CxA), SA vs CxA, and the like. The embryo ploidy classification or prediction can provides clinically actionable information, as it can inform the determination of which embryo to select for implantation and thus provides clinically actionable information, in an improved and non-invasive manner, which has the further advantages of not being subject to human bias or error.

The time point analyses show that among various Day 1-5 models developed and tested, Day 5 of embryo development provides the most information for ploidy discrimination. As such, image frames used in the model described herein can be taken 96-112 hours post insemination (hpi). The data type comparison found that video classification models provided better performance than image classification models, thus demonstrating that temporal information from additional time points provides models with heightened ability to determine ploidy status. Moreover, video classification models are more consistent across different embryo datasets than single image models, owing to added temporal information. The model-derived video-based blastocyst score and ploidy classification model described herein outperforms both image- and prior video-based ploidy models while not requiring any subjective embryologist input.

The studies described herein additionally show that the inclusion of maternal age as a parameter in the analyses can enhance the output result and can improve performance over analysis based on video data alone. Thus, the strongest models considered herein were based on datasets which include Day 5 video (image frames) as well as maternal age.

Because this model is non-invasive and requires no manually-curated features, iterations and embodiments of this model can be adopted to clinical practice. This demonstrates the ability for the presently described model-derived video-based blastocyst score and ploidy classification model to be used alone or as a standardized supplementation to traditional (i.e. exclusively human, non-automated) methods of predicting euploid and single aneuploid embryos, and/or for embryo selection and prioritization for implantation or recommendation for pre-implantation genetic testing for aneuploid (PGT-A) evaluation.

Accordingly, the embodiments disclosed herein are generally directed towards systems, software and methods for evaluating embryos by, for example, predicting embryo ploidy status.

### II. Exemplary Descriptions of Terms

Unless otherwise defined, all terms of art, notations, and other scientific terms or terminology used herein are intended to have the meanings commonly understood by those of skill in the art to which this application pertains. Further, unless otherwise required by context, singular terms shall include pluralities and plural terms shall include the singular. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in various embodiments.

In addition, as the terms “on”, “attached to”, “connected to”, “coupled to”, or similar words are used herein, one element (e.g., a material, a layer, a substrate, etc.) can be “on”, “attached to”, “connected to”, or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, the terms “comprise”, “comprises”, “comprising”, “contain”, “contains”, “containing”, “have”, “having”, “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein the specification, “a”, “an”, and “the,” may mean one or more. These terms generally refer to singular and plural references unless the context clearly dictates otherwise. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined. “A and/or B” is used herein to include all of the following alternatives: “A”, “B”, “A or B”, and “A and B”.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, a “subject” or an “individual” includes animals, such as human (e.g., human individuals) and non-human animals. The term “non-human animals” includes all vertebrates, e.g., mammals, e.g., rodents, e.g., mice, non-human primates, and other mammals, such as e.g., rat, mouse, cat, dog, cow, pig, sheep, horse, goat, rabbit; and non-mammals, such as amphibians, reptiles, etc. A subject can be a mammal, preferably a human or humanized animal. The subject may be in need of prevention and/or treatment of a disease or disorder, such as infertility.

The term “patient,” as used herein, generally refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

“Treating” or treatment of a disease or condition refers to executing a protocol, which may include administering one or more drugs to an individual, such as a patient (or subject), in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition, such as infertility. In addition, “treating” or “treatment” does not require complete alleviation of signs or symptoms, does not require a cure, and specifically includes protocols that have only a marginal effect on the patient.

The term “therapeutically effective” as used throughout this application refers to anything that promotes or enhances the well-being of the subject with respect to the medical treatment of this condition. In some embodiments, administering a therapeutically effective amount results in treating the condition to some degree.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

Similarly, the terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. Biological samples may include, but are not limited to stool, synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to stool, biopsy, blood and/or plasma. In some examples, biological samples include, but are not limited, to urine or stool. Biological samples include, but are not limited, to biopsy. Biological samples include, but are not limited, to tissue dissections and tissue biopsies. Biological samples include, but are not limited, any derivative or fraction of the aforementioned biological samples. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “marker” or “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, markers or biomarkers may be used for diagnostic purposes (e.g., to diagnose a health state, a disease state). The term “biomarker” can be used interchangeably with the term “marker.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In various embodiments, the term “about” indicates the designated value ±up to 10%, up to ±5%, or up to ±1%.

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

It should be understood that while deep learning may be discussed in conjunction with various embodiments herein, the various embodiments herein are not limited to being associated only with deep learning tools. As such, machine learning and/or artificial intelligence tools generally may be applicable as well. Moreover, the terms deep learning, machine learning, and artificial intelligence may even be used interchangeably in generally describing the various embodiments of systems, software and methods herein.

In various embodiments, a deep learning, machine learning, and/or artificial intelligence system can take the form of one or more binary classification model. The binary classification model may include, for example, but is not limited to, a regression model. The binary classification model may include, for example, a penalized multivariable regression model that is trained to identify set of embryo features from a otherwise defined, scientific and technical plurality of (or panel of) identified embryo feature options. The binary classification model may be trained to identify weight coefficients for embryo features, and those embryo features having non-zero weights or weight coefficients above a selected threshold (e.g., absolute weight coefficient above 0.0, 0.01, 0.05, 0.1, 0.015, 0.2, etc.) may be selected for inclusion in the set of embryo features.

### III. Predicting Embryo Quality Based on Probability of Embryo Ploidy Status

Since its introduction in 1978, in vitro fertilization (IVF) has become the best option for infertile parents looking to conceive children. Over 9 million IVF children have been born, resulting from around 500,000 deliveries annually in recent years.¹ IVF begins with mature oocyte retrieval and insemination in vitro on Day 0. This leads to formation of an embryo, beginning Day 1 of embryo development. Embryos subsequently undergo frequent cell divisions. This continues until Day 5 or 6, where the embryo is at a suitable stage for implantation. After Day 5, embryos are graded for quality by embryologists. Embryos that score high are then selected for transfer into the mother.^{2,3} While some clinics opt to transfer the embryo on Day 3, transfer on Day 5 at the blastocyst stage is preferred as a means of selecting embryos with higher implantation potential and reducing the multiple pregnancy risk.

Ploidy status, which is the presence, or lack thereof, of chromosomal abnormalities is an important factor in a successful pregnancy. Embryos with no abnormalities are classified as euploidy and are generally correlated with successful pregnancies. Aneuploidy embryos include those where there are extra or missing chromosomes, leading to diseases such as Down Syndrome or Turner's Syndrome. A majority of miscarriages and unsuccessful pregnancies are due to aneuploid embryos. Advanced maternal age has also been found to be associated with a higher embryo aneuploidy rate.

To determine ploidy status of embryos, the current diagnostic method is a preimplantation genetic test for aneuploidy (PGT-A). Generally, small portions of the trophectoderm (TE) are biopsied, amplified and tested for chromosome copy number variants. Although PGT-A increases implantation rate by addressing the issue of embryo aneuploidy and is a more standardized method of evaluating embryos, several limitations remain.⁴ PGT-A is an expensive and time-consuming process. Also, PGT-A is an invasive test, and biopsy can impair embryo viability. PGT-A is also susceptible to false negative and false positive results from a phenomenon known as embryonic mosaicism, in which both aneuploid and euploid cells co-exist in the TE, which may result in a reduction of embryo quality and viability.⁵

With the advent of artificial intelligence in the field of computer vision and the collection of large datasets of IVF-related datasets combining images, videos and clinical outcomes, a variety of methods have been developed to automatically access embryo quality and other characteristics using images from time-lapse sequences. For example, Khosravi et al. have developed STORK, an embryo morphological assessment model which predicted embryo quality accurately, where these predictions were correlated with positive birth outcomes.⁶ Similar algorithms can be applied to predict embryo ploidy, as embryo images can contain patterns or information that are characteristic of chromosomal abnormalities. For example, Barnes et al. have developed machine learning algorithms to predict ploidy status of embryos based on a single image at 110-h.⁷ These algorithms use single time-point images extracted from time-lapse videos.

Previous attempts have been made to leverage entire video sequences to improve embryo classification accuracy. For example, Silver et al. created a CNN-LSTM model named UBar, which was trained on 272 embryo videos. This model was trained to differentiate between successful and unsuccessful implantations and had an AUC of 0.82, albeit on a limited dataset.⁸ A recent investigation done by Lee et al. presented a 2-stream inflated 3d (I3D) model trained on 670 videos to discriminate between euploid/mosaic and aneuploidy embryos, achieving an AUC of 0.74.⁹

A problem that arises from analyzing entire time-lapse sequences of embryos, as in the examples mentioned above, is that not all stages have relevant information for ploidy status prediction. Thus, previous studies, including those mentioned above, have opted to extract features from specific periods of embryo development. Campbell et al. suggested that presence and time to blastocyst expansion (Day 5) can predict ploidy status.¹⁰ However, Campbell's criteria have been shown to vary in prediction power across clinics, and this work has been contested.¹¹ Other investigations have opted to use time-lapse images only at Day 4 of development to build prediction models, with limited success. A thorough investigation into which days of embryo development provide the most discriminatory information for ploidy status has heretofore been lacking, and previous models developed to date have been either indiscriminate (video models) or insufficiently predictive and/or reproducible (Day 4 and earlier models).

To address this gap, various ploidy status prediction models were developed, and their performance was compared across different stages of embryo development, as described herein. Based on model performance, it was determined which periods of embryo development provide information which is important for detecting ploidy status. In addition to time point analyses, prediction model performances were compared between image and video classification. By doing so, this investigation determined whether adding temporal information from sequences of time-lapse images provides any advantage in terms of classification performance. This is the first study assessing both image and video classification tasks as well as exploring which timepoints contribute most significantly to classification. Through this study, a completely automated ploidy prediction model has been developed, namely the model-derived video-based blastocyst score and ploidy classification model, where the inputs include embryo time-lapse sequence and maternal age. By eliminating subjective manual annotation, the model-derived video-based blastocyst score and ploidy classification model can be deployed universally across different clinical settings can may act as a supporting tool for embryologist decision-making.

Previous studies on IVF embryo classification have generally used training data from later stages of embryo development and only focused on either image or temporally indiscriminate video data. In this study, several deep learning models were developed to discriminate embryo ploidy status as euploidy or a form of aneuploidy. The performances of deep learning models across different time periods were also compared. By doing so, the study investigated whether utilizing only late stage data from time-lapse imaging sequences is warranted. Performance differences between models trained on image data versus video data were also evaluated to determine whether added temporal information within videos aids in ploidy discrimination.

The results presented herein demonstrate that Day 5 of embryo development provides the most information for ploidy discrimination. Day 5 models were found to perform significantly better than All Day models as well (see Example 2, Table 2); this demonstrates that information, or lack thereof, detracts from model performance. In essence, features extracted from Day 1-4 may not be informative and can decrease overall model performance in All Day models, as compared to Day 5 models. This is consistent with the biological nature of embryo development, as specific features associated with chromosomally normal embryos, such as cavitation, occur on Day 5.¹⁰

Other studies have suggested that other morphological characteristics of the embryo that occur early in development, such as rate of cleavage, cell count, and length of morula stage, can be suggestive of a “successful” embryo.¹⁴ However, many of these studies have different definitions of what a “successful” embryo is, varying from an embryo that reaches the blastocyst stage to an embryo that leads to a pregnancy. While a chromosomally normal embryo may be correlated with these success criteria, the indicators may not necessarily be the same.

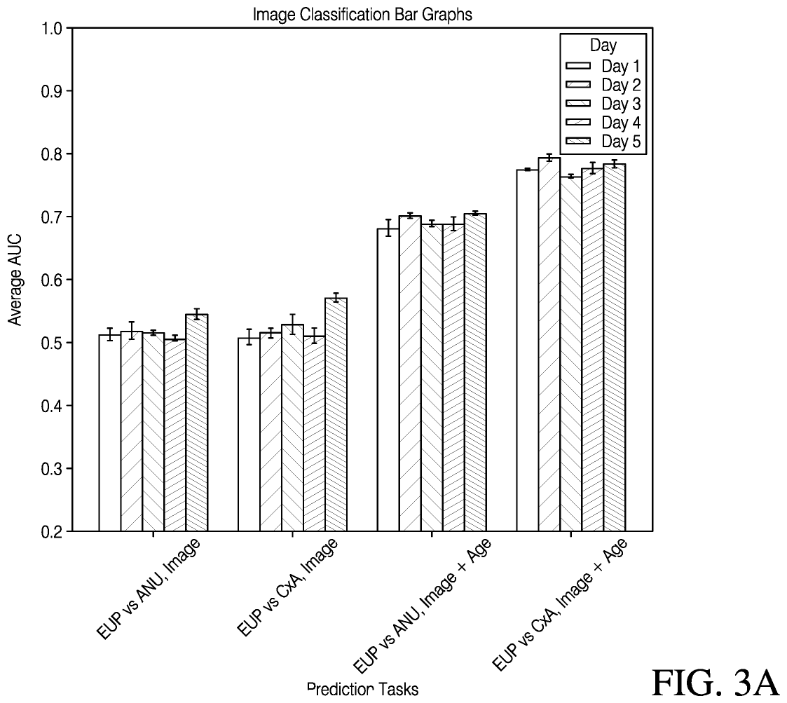

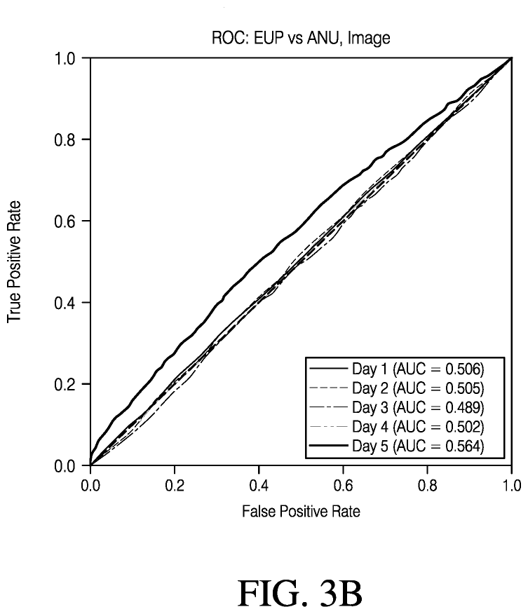

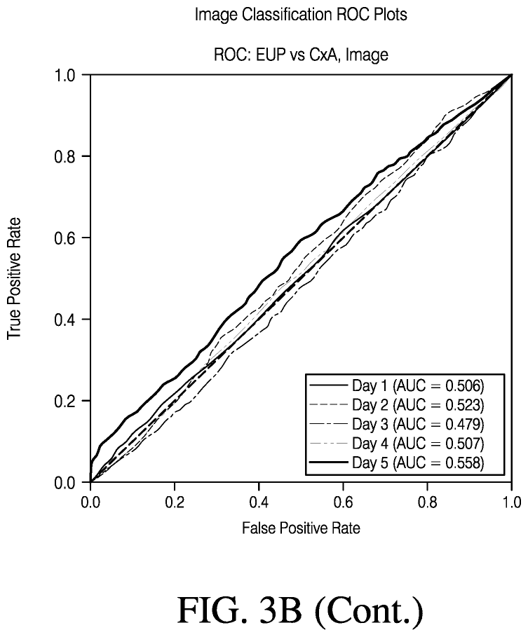

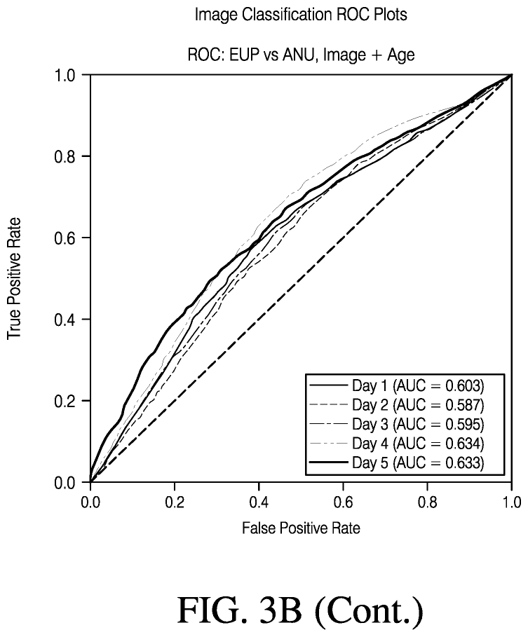

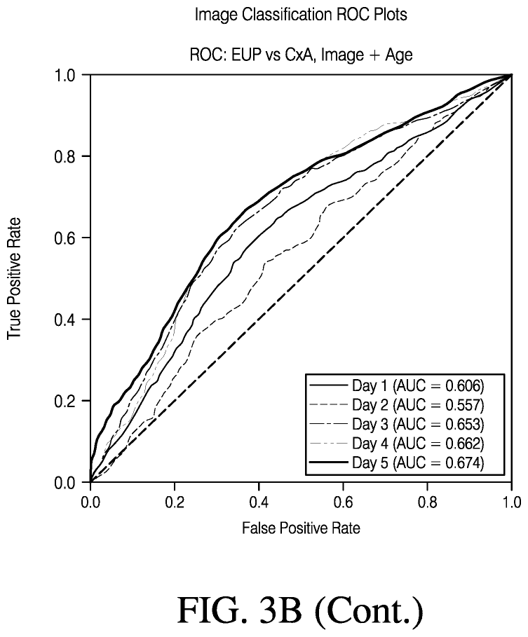

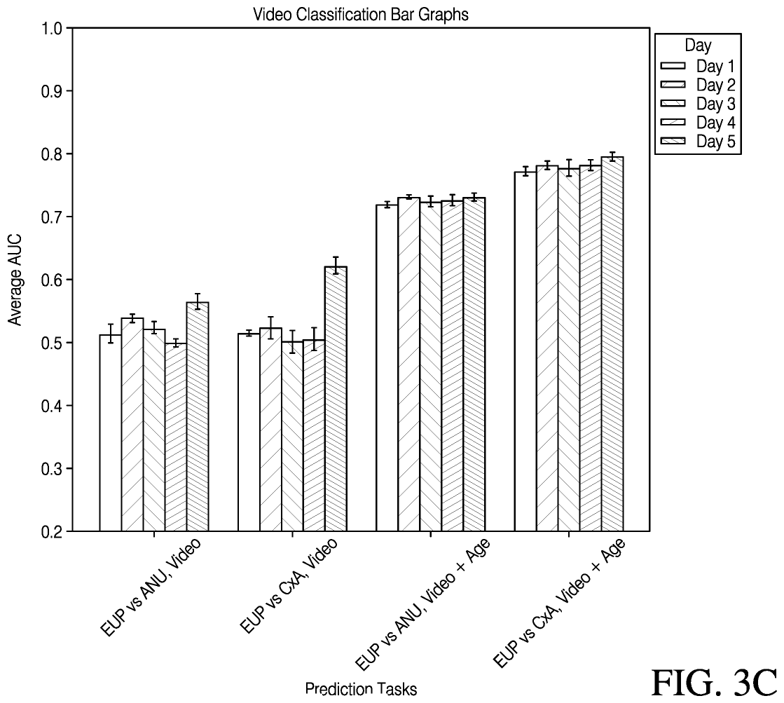

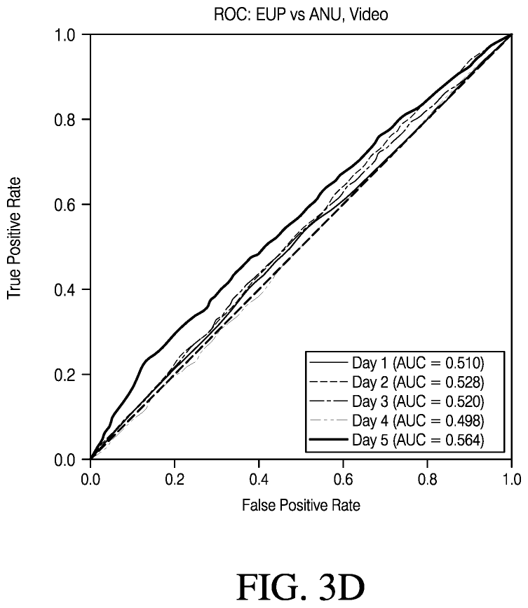

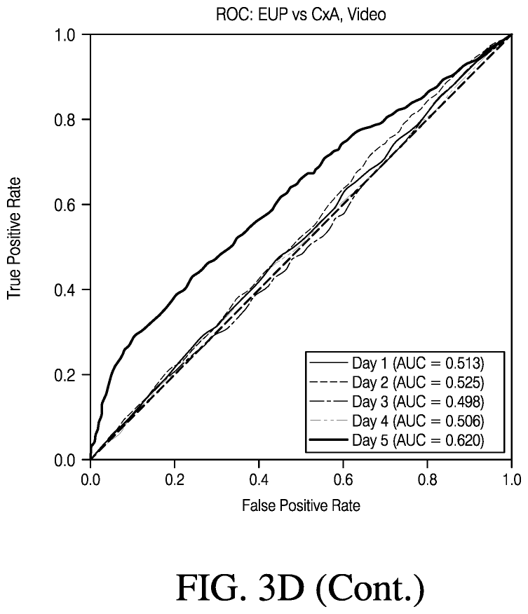

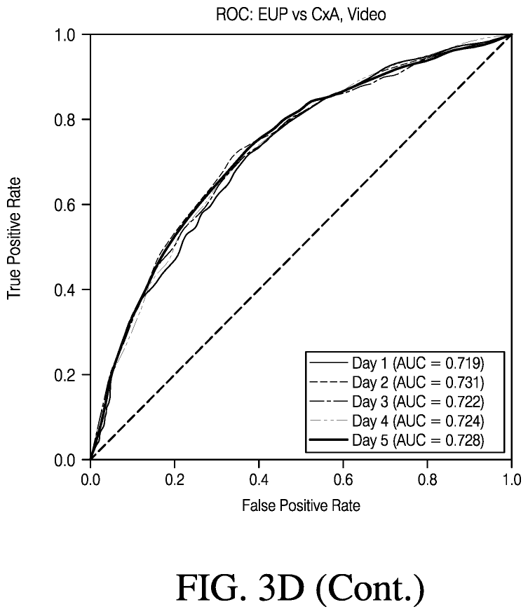

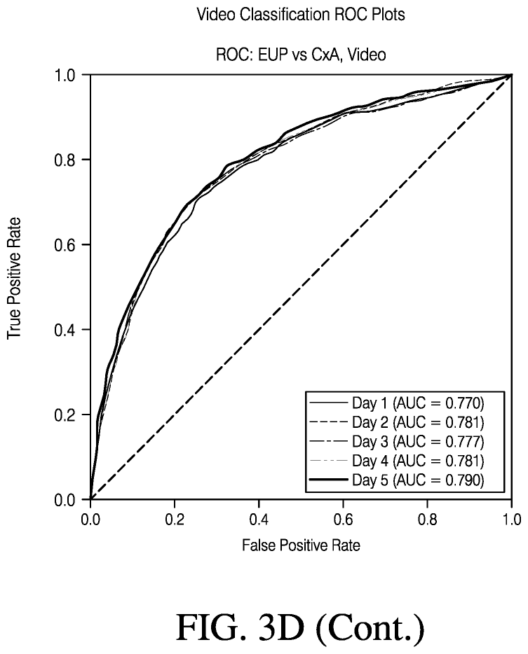

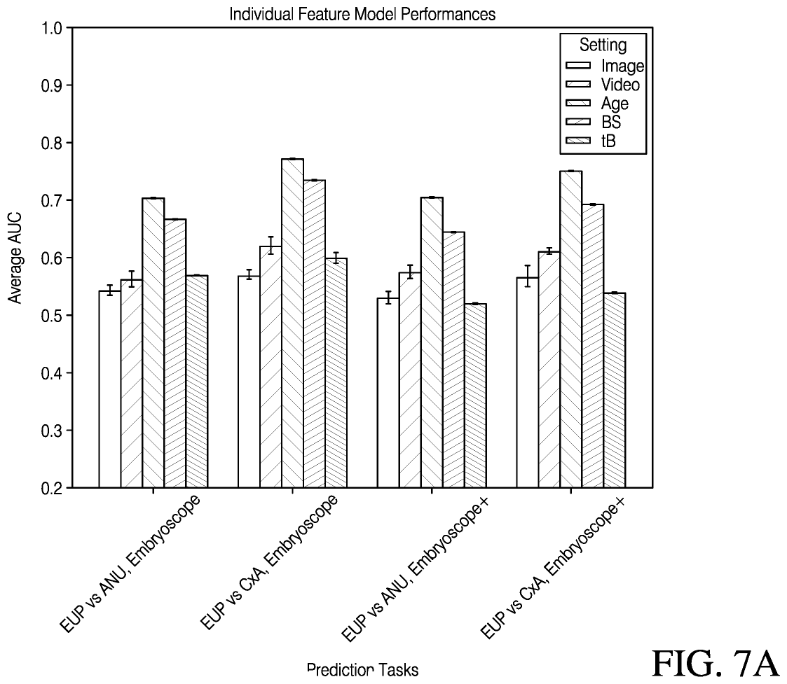

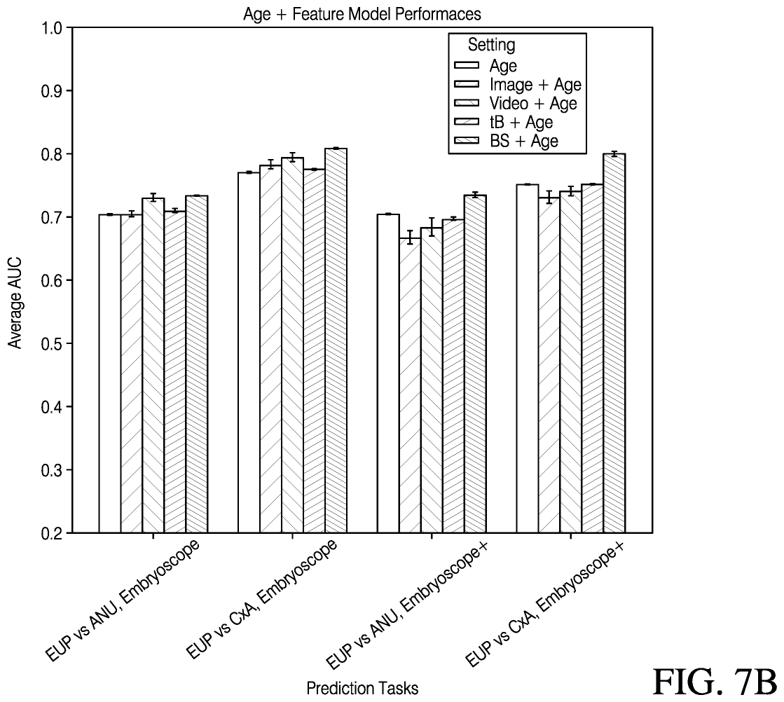

The present study shows that these early indicators of embryo “success” are not suggestive of ploidy status. Interestingly, when maternal age was added as a feature to the models, there were no significant differences across time periods (see Example 2, FIGS. 3A-3D). Performance across all prediction tasks and datasets were higher with the addition of maternal age. This demonstrates that maternal age is a strong predictor of ploidy status, which is consistent with previous studies, and stronger than both video and image data (see Example 3, FIGS. 7A-7B).

The second analysis described herein compared Day 5 image classification models to video classification models. Video-only models performed better than image-only models at all prediction tasks on both the Embryoscope and Embryoscope+ datasets. Video+age models also performed significantly better than image+age models in all prediction tasks on Embryoscope data. These results desmonstrate that the temporal information from additional frames provides models with heightened ability to classify embryos by their ploidy status.

BiLSTM models, which are able to extract this temporal information, perform significantly better on video data than XGBoost models (see Example 3, Table 5). This further demonstrates that temporal relations between frames plays a role in the significant performance increase between image and video models. Moreover, video classification models, while suffering from performance decreases when transferring from one dataset to another, can be more consistent across different embryo datasets, owing to its decreased variability to added temporal information. Therefore, the rate of cavitation and speed to blastulation can be markers for ploidy that the presently described video classification models are able to extract (see Example 3, FIG. 7A). Complete time-lapse image sequences are more difficult to curate and process than a single time-lapse image, which adds relatively more computational overhead to video classification models. However, the added temporal information from video data allows for models to be transferable across clinics without performance deterioration and therefore warrants the computational overhead.

Lastly, a fully automated model is described herein, wherein blastocyst score is predicted, and the predictions are used as a proxy for ploidy classification. The model-derived video-based blastocyst score and ploidy classification model performs comparably to a model trained on the embryologist-annotated blastocyst score, and it does significantly better than models trained only on time-lapse imaging sequences (without a proxy score). Moreover, the model-derived video-based blastocyst score and ploidy classification model is completely automated, and includes time-lapse images from 96 hpi-112 hpi and maternal age to predict ploidy status of embryo. This allows the model-derived video-based blastocyst score and ploidy classification model to be adapted clinically without interrupting ongoing workflows.

The performance of the model-derived video-based blastocyst score and ploidy classification model was also compared to a single-image-based ploidy classification model (trained on 108 hpi images). The model-derived video-based blastocyst score and ploidy classification model performed significantly better, further showing that added information from different frames increases prediction power. The model-derived video-based blastocyst score and ploidy classification model also provides a certain level of explainability; i.e. embryologists can use the model-derived video-based blastocyst score and ploidy classification model (along with the other scores predicted via multitasking) to determine reasons as to why an embryo is classified as a certain ploidy status. With a recall of around ˜0.85 for Embryoscope+dataset, the model-derived video-based blastocyst score and ploidy classification model can successfully select for euploid embryos (see Example 4, Table 7).

Interestingly, the application of the euploid (EUP) vs complex aneuploidy (CxA) successfully on simple aneuploid (SA) embryos evenly predicts them as EUP or CxA. This result is consistent with the idea that simple aneuploidy embryos are more difficult to identify and can often look either like euploid or complex aneuploid embryos. Models like the model-derived video-based blastocyst score and ploidy classification model del can therefore provide supplemental information in embryologist decision-making.

In summary, several machine learning models that require no embryologist-derived features have been developed. Day 5 models have been shown to provide significantly more information than other day models in determining ploidy status, and video classification models have been demonstrated to provide significantly better performance and transferability as compared to image classification models. Future iterations of these model-derived video-based blastocyst score and ploidy classification models can therefore be adopted to clinical practice because they require no manually-curated features and are from end-to-end, fully automated. The models developed as described herein are clinically relevant in making decisions on whether an embryo is chromosomally normal. Moreover, automated blastocyst score prediction models can be clinically relevant to embryologists who are currently annotating embryo scores manually. In the case where the model-derived video-based blastocyst score and ploidy classification model is not used end-to-end to predict embryo ploidy, it can be used to supplement manual embryo quality scoring. Unlike many previous investigations that have used arrested/non-viable embryos as negatives and resulted in higher AUCs, the present data only consists of good biopsied embryos. As such, the resulting models are much more powerful, as well as clinically applicable.

### IV. Overview of Exemplary Workflow

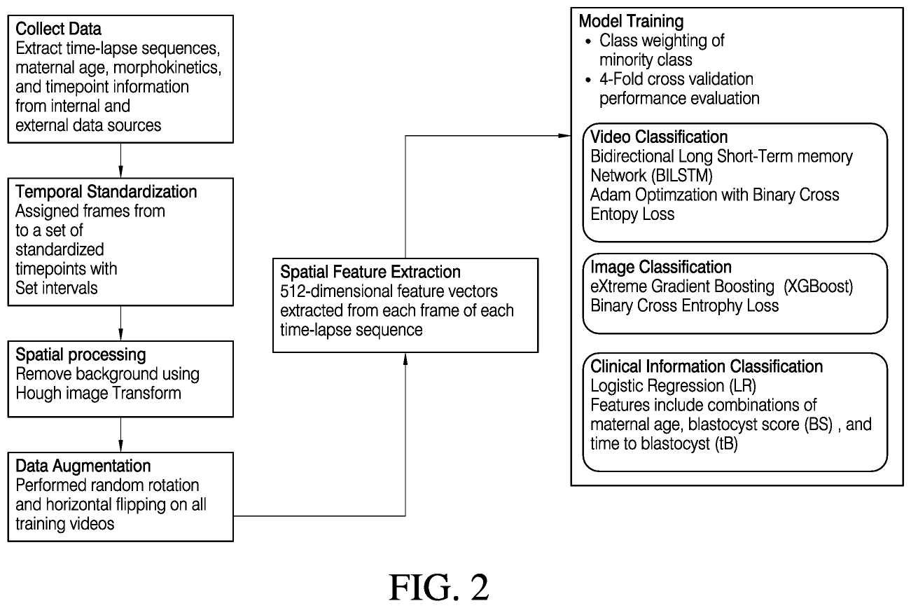

An exemplary workflow for various embodiments in accordance with the present disclosure, used for predicting ploidy status of an embryo, for example such as during in vitro fertilization, is shown in FIG. 2. This exemplary workflow utilizes the presently described model-derived blastocyst score (MDBS) and ploidy classification model. Specific details can be found in Example 2.

FIG. 2 depicts the following steps: 1) data collection; 2) temporal standardization; 3) spatial processing; 4) data augmentation; 5) spatial feature extraction; and 6) model training (e.g. via video classification, image classification, and/or clinical information classification). First, as shown in step 1, data associated with an embryo can be collected. For example, a dataset including at least two or more time-lapse image frames from a video obtained for an embryo are analyzed. Optionally, the dataset can contain data relating to one or more morphokinetic annotations, morphological assessments, maternal age, and/or associated PGT-A results for the embryo can be included in the analysis. As shown in step 2, he two or more time-lapse image frames can then be assigned to a set of standardized timepoints with set intervals. Optionally, as shown in step 3, the dataset can be pre-processed and/or spatially processed, such as to remove background (e.g. using Hough Image Transform), to remove underexposed images by manual detection, and/or to input missing image and/or morphokinetic values using median imputation. Optionally, as shown in step 4, the data can then be augmented or otherwise modified as appropriate, such as by performing random rotation and horizontal flipping on all training videos. As shown in step 5, spatial features can then be extracted from the pre-processed, spatially processed, and/or augmented data. For example, spatial feature extraction can be performed by extracting 512-dimensional feature vectors from each frame of each time-lapse sequence.

As shown in step 6, the model can then be trained via class weighting of the minority class, using, e.g., 4-fold cross validation for performance evaluation. This includes video classification, image classification, and clinical information classification. Video classification can be performed using, e.g., Bidirectional Long Short-Term Memory Network (BiLSTM) and/or Adam Optimization with Binary Cross Entropy Loss. Image classification can be performed using, e.g., eXtreme Gradient Boosting (XGBoost) Binary Cross Entropy Loss. Clinical information can be classified using, e.g., LASSO and logistic regressions to determine feature importance. Features include, e.g., combinations of maternal age, blastocyst score (BS), and time to blastocyst (tB). Hyperparameters for the models can then be optimized through iterative training and once completed, the performance on the test set can be evaluated. One or more deep learning model can be used for ploidy classification, where extracted image features can be concatenated with clinical information (e.g., maternal age, one or more morphokinetic parameters, and/or one or more morphological assessments, such as blastocyst grade (BG), blastocyst score (BS), time to blastocyst (tB), artificial intelligence-driven predicted blastocyst score (AIBS) before being passed on to a final fully-connected layer.

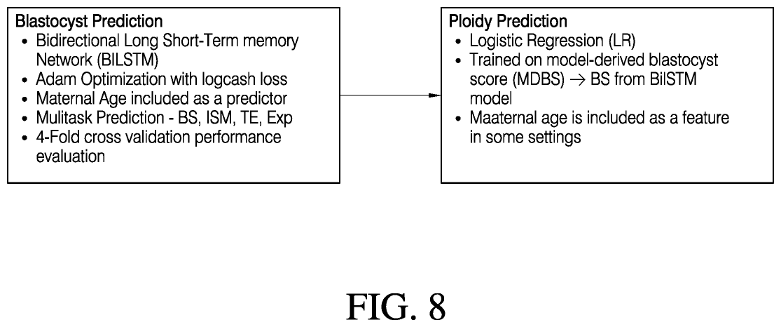

FIG. 8 depicts an exemplary workflow for generating a blastocyst prediction using the model as training as shown in FIG. 2, and translating the blastocyst prediction into a ploidy prediction. Further detail can be found in Example 4.

As shown in FIG. 8, features can be extracted from time-lapse image frames as shown in FIG. 2, steps 1-5. These features can be fed into a multitask Bidirectional Long Short-Term Memory (BiLSTM) model which is trained to predict blastocyst score as well as other embryologist-annotated morphological scores, and predicted blastocyst scores can then be inputted into a logistic regression model to perform ploidy classification. First, for example, a (BiLSTM) network is used, optionally along with Adam Optimization with log cosh loss. Maternal age can be included as a predictor. A multitask prediction is generated, based on determination of BS, inner-cell mass (ICM) grade, trophectoderm (TE) grade, and/or expansion score. Performance can be evaluated via 4-fold cross validation.

Ploidy prediction can be achieved via logistic regression. The ploidy prediction model is trained on the model-derived blastocyst score, which includes the blastocyst score generated from the BiLSTM model. Maternal age can be included as a feature in some settings.

These examples are described for illustrative purposes only, and other workflows are contemplated in accordance with various embodiments, involving additional steps and/or features, and/or removing certain steps and/or features used in illustrative exemplary embodiments.

The workflow may include various operations including, for example, sample collection, sample intake, sample preparation and processing, data analysis, and output generation.

Sample collection may include, for example, obtaining a biological sample of one or more subjects. The biological sample may take the form of a specimen obtained via one or more sampling methods. The biological sample may be a sample taken to obtain maternal, paternal, and/or embryonic genetic information. The biological sample may be obtained in any of a number of different ways. In various embodiments, the biological sample includes whole blood sample obtained via a blood draw. In various embodiments, the biological sample includes a cryopreserved whole blood sample or a cryopreserved sample. In other embodiments, the biological sample includes a set of aliquoted samples that includes, for example, a serum sample, a plasma sample, a blood cell (e.g., white blood cell (WBC), red blood cell (RBC)) sample, another type of sample, or a combination thereof. Biological samples may include nucleotides (e.g., ssDNA, dsDNA, RNA), organelles, amino acids, peptides, proteins, carbohydrates, glycoproteins, or any combination thereof.

Sample intake may include one or more various operations such as, for example, aliquoting, registering, processing, storing, thawing, and/or other types of operations.

Further, sample preparation and processing may include, for example, data acquisition based on a video including a plurality (i.e. two or more) image frames of an embryo. Sample preparation and processing may also include, for example, data acquisition based on clinical and/or morphological features for the embryo.

Data analysis may include, for example, machine and/or deep learning and regression analysis of a video including a plurality (i.e. two or more) image frames of an embryo and/or clinical and/or morphological features for the embryo. In some embodiments, data analysis also includes output generation. In other embodiments, output generation may be considered a separate operation from data analysis. Output generation may include, for example, generating final output based on the results of machine and/or deep learning and regression analysis of a video including a plurality (i.e. two or more) image frames of an embryo and/or clinical and/or morphological features for the embryo. In various embodiments, final output may be used for determining embryo ploidy. In various embodiments, final output may be used for determining the research, diagnosis, and/or treatment of infertility, by predicting embryo viability based on the embryo ploidy status, wherein an embryo having a stronger probability of being euploid has a higher probability of being viable. In various embodiments, final output may be used for determining the research, diagnosis, and/or treatment of infertility, by improving embryo selection during in vitro fertilization. In various embodiments, final output may be used for determining the research, diagnosis, and/or treatment of infertility, by selecting and/or prioritizing an embryo for preimplantation genetic testing for aneuploidy (PGT-A) biopsy and/or implantation during in vitro fertilization. In various embodiments, final output may be used for determining the research, diagnosis, and/or treatment of infertility, by combining the results of machine and/or deep learning and regression analysis of a video including a plurality (i.e. two or more) image frames of an embryo and/or clinical and/or morphological features for the embryo with traditional methods of embryo selection and prioritization for implantation and/or recommendation for PGT-A during in vitro fertilization. In various embodiments, final output may be used for determining the research, diagnosis, and/or treatment of infertility, by improving an outcome in a subject undergoing in vitro fertilization, comprising the method of any preceding claim, wherein an embryo predicted to be euploid is selected for embryo transfer during in vitro fertilization.

In various embodiments, final output is comprised of one or more outputs. Final output may take various forms. In some embodiments, the final output can include a report. For example, final output may be a report that includes, for example, a diagnosis output, a treatment output (e.g., a treatment design output, a treatment plan output, or combination thereof), analyzed data (e.g., relativized and normalized) or combination thereof.

In some embodiments, the final output can include a report having a probability of the ploidy status of the embryo analyzed, also referred to herein as embryo ploidy. For example, the report can include a probability of the embryo being euploid (EUP) vs aneuploid (ANU), EUP vs simple aneuploid (SA), EUP vs. complex aneuploid (CxA), SA vs CxA, and the like. The embryo ploidy prediction can be used to determine which embryo to select for implantation and thus provides clinically actionable information. For example, a report stating a high probability of the embryo being euploid can indicate that the embryo may be suitable for implantation. In contrast, a report stating a high probability of the embryo being aneuploid (e.g. simple aneuploid or complex aneuploid) can indicate that the embryo may be less suitable for implantation, as compared to an embryo with a high probability of being euploid. Further, a report stating a high probability of the embryo being simple aneuploid (as opposed to complex aneuploid) can indicate that the embryo may be suitable for implantation.

In some embodiments, a high probability indicates a probability greater than 50%. In some embodiments, a high probability indicates a probability greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or higher.

In some embodiments, final output may be sent to a remote system for processing. The remote system may include, for example, a computer system, a server, a processor, a cloud computing platform, cloud storage, a laptop, a tablet, a smartphone, some other type of mobile computing device, or a combination thereof. In other embodiments, a final output may be displayed on a graphical user interface in a display system for viewing by a human operator.

In other embodiments, any workflow as described herein may optionally exclude one or more of the operations described herein and/or may optionally include one or more other steps or operations other than those described herein (e.g., in addition to and/or instead of those described herein). Accordingly, any workflow as described herein may be implemented in any of a number of different ways to determine a probability of embryo ploidy and/or for use in the research, diagnosis, and/or treatment of, for example, infertility.

### V. Neural Networks

Image classification/recognition generally requires accepting an input image and outputting a class or a probability of classes that best describes the image. This can be done using a computer system equipped with a processing engine, which utilizes algorithms, to process the input image and outputting a result. Image detection can also utilize a similar processing engine, whereby the system accepts an input image and identifies objects of interest within that image with a high level of accuracy using the algorithms pre-programmed into the processing engine.

Regarding the input image, the system will generally orient the input image as an array of pixel values. These pixel values, depending on the image resolution and size, will be an array of numbers corresponding to (length)×(width)×(# of channels). The number of channels can also be referred to as the depth. For example, the array could be L×W×Red Green Blue color model (RBG values). The RGB would be considered three channels, each channel representing one of the three colors in the RGB color model. For example, the system can generally characterize a 20×20 image with a representative array of 20×20×3 (for RGB), with each point in the array assigned a value (e.g., 0 to 255) representing pixel intensity. Given this array of values, the processing engine can process these values, using its algorithms, to output numbers that describe the probability of the image being a certain class (e.g., 0.80 for cell, 0.15 for cell wall, and 0.05 for no cell).

A deep neural network (DNN) generally, such as a convolutional neural network (CNN), generally accomplishes an advanced form of image processing and classification/detection by first looking for low level features such as, for example, edges and curves, and then advancing to more abstract (e.g., unique to the type of images being classified) concepts through a series of convolutional layers. A DNN/CNN can do this by passing an image through a series of convolutional, nonlinear, pooling (or downsampling, as will be discussed in more detail below), and fully connected layers, and get an output. Again, the output can be a single class or a probability of classes that best describes the image or detects objects on the image.

Regarding layers in a CNN, for example, the first layer is generally a convolutional layer (Conv). This first layer will process the image's representative array using a series of parameters. Rather than processing the image as a whole, a CNN will analyze a collection of image sub-sets using a filter (or neuron or kernel). The sub-sets will include a focal point in the array as well surrounding points. For example, a filter can examine a series of 5×5 areas (or regions) in a 32×32 image. These regions can be referred to as receptive fields. Since the filter must possess the same depth of the input, an image with dimensions of 32×32×3 would have a filter of the same depth (e.g., 5×5×3). The actual step of convolving, using the exemplary dimensions above, would involve sliding the filter along the input image, multiplying filter values with the original pixel values of the image to compute element wise multiplications, and summing these values to arrive at a single number for that examined portion of the image.

After completion of this convolving step, using a 5×5×3 filter, an activation map (or filter map) having dimensions of 28×28×1 will result. For each additional layer used, spatial dimensions are better preserved such that using two filters will result in an activation map of 28×28×2. Each filter will generally have a unique feature it represents (e.g., colors, edges, curves, etc.) that, together, represent the feature identifiers required for the final image output. These filters, when used in combination, allow the CNN to process an image input to detect those features present at each pixel. Therefore, if a filter serves as a curve detector, the convolving of the filter along the image input will produce an array of numbers in the activation map that correspond to high likelihood of a curve (high summed element wise multiplications), low likelihood of a curve (low summed element wise multiplications) or a zero value where the input volume at certain points provided nothing that would activate the curve detector filter. As such, the greater number of filters (also referred to as channels) in the Conv, the more depth (or data) that is provided on the activation map, and therefore more information about the input that will lead to a more accurate output.

Balanced with accuracy of the CNN is the processing time and power needed to produce a result. In other words, the more filters (or channels) used, the more time and processing power needed to execute the Conv. Therefore, the choice and number of filters (or channels) to meet the needs of the CNN method are specifically chosen to produce as accurate an output as possible while considering the time and power available.

To enable further a CNN to detect more complex features, additional Conv layers can be added to analyze what outputs from the previous Conv layer (i.e., activation maps). For example, if a first Conv layers looks for a basic feature such as a curve or an edge, a second Conv layer can look for a more complex feature such as shapes, which can be a combination of individual features detected in an earlier Conv layer. By providing a series of Conv layers, the CNN can detect increasingly higher-level features to arrive eventually at the specific desired object detection. Moreover, as the Conv layers stack on top of each other, analyzing the previous activation map output, each Conv layer in the stack is naturally going to analyze a larger and larger receptive field by virtue of the scaling down that occurs at each Conv level, thereby allowing the CNN to respond to a growing region of pixel space in detecting the object of interest.

A CNN architecture generally consists of a group of processing blocks, including at least one processing block for convoluting an input volume (image) and at least one for deconvolution block (or transpose convolution). Additionally, the processing blocks can include at least one pooling block and unpooling block. Pooling blocks can be used to scale down an image in resolution to produce an output available for Conv. This can provide computational efficiency (efficient time and power), which can in turn improve actual performance of the CNN. Those these pooling, or subsampling, blocks keep filters small and computational requirements reasonable, these blocks coarsen the output (can result in lost spatial information within a receptive field), reducing it from the size of the input by a factor equal to the pixel stride of the receptive fields of the output units.

Unpooling blocks can be used to reconstruct a these coarse outputs to produce an output volume with the same dimensions as the input volume. An unpooling block can be considered a reverse operation of a convoluting block to return an activation output to the original input volume dimension.

However, the unpooling process generally just simply enlarges the coarse outputs into a sparse activation map. To avoid this result, the deconvolution block densifies this sparse activation map to produce both and enlarged and dense activation map that eventually, after any further necessary processing, a final output volume with size and density much closer to the input volume. As a reverse operation of the convolution block, rather than reducing multiple array points in the receptive field to a single number, the deconvolution block associate a single activation output point with a multiple outputs to enlarge and densify the resulting activation output.

It should be noted that while pooling blocks can be used to scale down an image and unpooling blocks can be used to enlarge these scaled down activation maps, convolution and deconvolution blocks can be structured to both convolve/deconvolve and scale down/enlarge without the need for separate pooling and unpooling blocks.

The pooling and unpooling process can be limited depending on the objects of interest being detected in an image input. Since pooling generally scales down an image by looking at sub-image windows without overlap of windows, there is a clear loss in spatial info as the scaling down occurs.

A processing block can include other layers that are packaged with a convolutional or deconvolutional layer. These can include, for example, a rectified linear unit layer (ReLU) or exponential linear unit layer (ELU), which are activation functions that examine the output from a Conv layer in its processing block. The ReLU or ELU layer acts as a gating function to advance only those values corresponding to positive detection of the feature of interest unique to the Conv layer its processing block.

Given a basic architecture, the CNN is then prepared for a training process to hone its accuracy in image classification/detection (of objects of interest). Using training data sets, or sample images used to train the CNN so that it updates its parameters in reaching an optimal, or threshold, accuracy, a process called backpropagation (backprop) occurs. Backpropagation involves a series of repeated steps (training iterations) that, depending on the parameters of the backprop, either will slowly or quickly train the CNN. Backprop steps generally include forward pass, loss function, backward pass, and parameter (weight) update according to a given learning rate. The forward pass involves passing a training image through the CNN. The loss function is a measure of error in the output. The backward pass determines the contributing factors to the loss function. The weight update involves updating the parameters of the filters to move the CNN towards optimal. The learning rate determines the extent of weight update per iteration to arrive at optimal. If the learning rate is too low, the training may take too long and involve too much processing capacity. If the learning rate is too fast, each weight update may be too large to allow for precise achievement of a given optimum or threshold.

The backprop process can cause complications in training, thus leading to the need for lower learning rates and more specific and carefully determined initial parameters upon start of training. One such complication is that, as weight updates occur at the conclusion of each iteration, the changes to the parameters of the Conv layers amplify the deeper the network goes. For example, if a CNN has a plurality of Conv layers that, as discussed above, allows for higher-level feature analysis, the parameter update to the first Conv layer is multiplied at each subsequent Conv layer. The net effect is that the smallest changes to parameters have large impact depending on the depth of a given CNN. This phenomenon is referred to as internal covariate shift.

It should be noted that even though CNNs are spoken about in detail above, the various embodiments discussed herein could utilize any neural network type or architecture.

### VI. Computer-Implemented System

In various embodiments, the systems and methods for determining embryo ploidy status can be implemented via computer software or hardware.

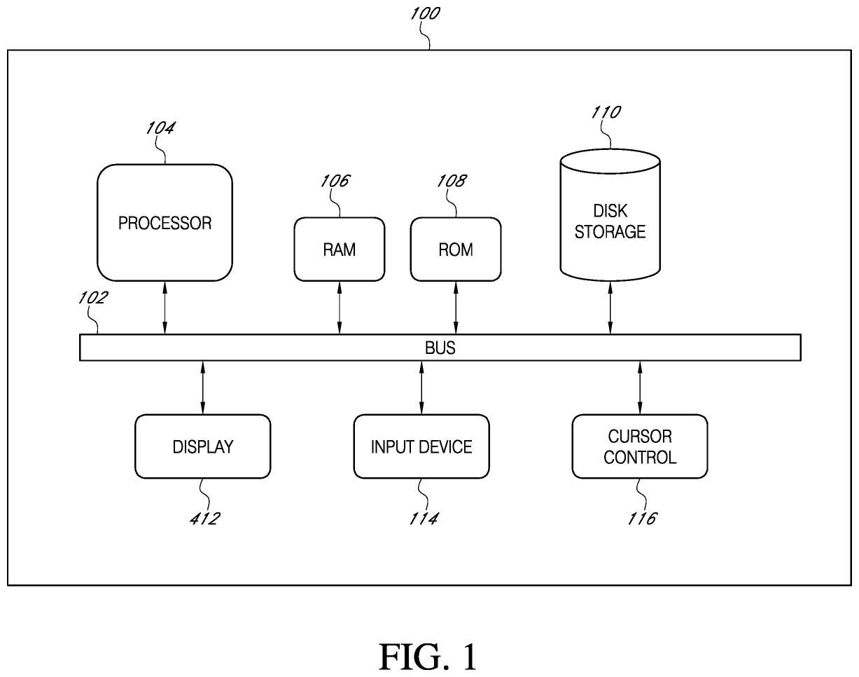

FIG. 1 is a block diagram illustrating a computer system 100 upon which embodiments of the present teachings may be implemented. In various embodiments of the present teachings, computer system 100 can include a bus 102 or other communication mechanism for communicating information and a processor 104 coupled with bus 102 for processing information. In various embodiments, computer system 100 can also include a memory, which can be a random-access memory (RAM) 106 or other dynamic storage device, coupled to bus 102 for determining instructions to be executed by processor 104. Memory can also be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communication of information and command selections to processor 104. Another type of user input device is a cursor control 116, such as a mouse, a trackball or cursor direction keys for communicating direction information and command selections to processor 104 and for controlling cursor movement on display 112. This input device 114 typically has two degrees of freedom in two axes, a first axis (i.e., x) and a second axis (i.e., y), that allows the device to specify positions in a plane. However, it should be understood that input devices 114 allowing for 3-dimensional (x, y and z) cursor movement are also contemplated herein.

Consistent with certain implementations of the present teachings, results can be provided by computer system 100 in response to processor 104 executing one or more sequences of one or more instructions contained in memory 106. Such instructions can be read into memory 106 from another computer-readable medium or computer-readable storage medium, such as storage device 110. Execution of the sequences of instructions contained in memory 106 can cause processor 104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 104 for execution. Such a medium can take many forms, including but not limited to, non-volatile media, volatile media, and transmission media. Examples of non-volatile media can include, but are not limited to, dynamic memory, such as memory 106. Examples of transmission media can include, but are not limited to, coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 104 of computer system 100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 100 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 100, whereby processor 104 would execute the analyses and determinations provided by these engines, subject to instructions provided by any one of, or a combination of, memory components 106/108/110 and user input provided via input device 114.

Although specific embodiments and applications of the disclosure have been described in this specification, these embodiments and applications are exemplary only, and many variations are possible. Having described the various embodiments in detail, it will be apparent that modifications, variations, and equivalent embodiments are possible without departing from the scope of the disclosure defined in the appended claims. Furthermore, it should be appreciated that all examples in the present disclosure are provided as non-limiting examples.

### VII. Examples

The following non-limiting examples are provided to further illustrate embodiments of the disclosure herein. It should be appreciated by those of skill in the art that the techniques disclosed in the examples that follow represent approaches that have been found to function well in the practice of the disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the disclosure.

### Example 1

### Methods

The methods used in Examples 2-5 are described below.

### Source Data

The first dataset for this study, i.e. Embryoscope data, includes time lapse images (800×800 pixels) and PGT-A results for 1998 embryos (SA n=494, CxA n=588, and EUP n=916) collected from the Center of Reproductive Medicine at Weill Cornell Medicine between 2018-2019 (IRB number: 1401014735 and 19-06020306). Clinical information such as embryologist-derived blastocyst score (BS), morphokinetic parameters, and maternal age at the time of oocyte retrieval were collected in addition to time-lapse sequences. Generally, most time lapse image sequences consisted of around 360-420 unique frames corresponding with 5 days of development with an image taken every 0.3 hours. The blastocyst score is the sum of a set of scores that are converted from the expansion, inner-cell mass (ICM), trophectoderm (TE) grades, and day of blastocyst formation.¹⁵ The blastocyst score ranges from 3-14, where a lower number is associated with a higher-quality embryo. This study used retrospective and fully de-identified data.

PGT-A results were categorized into two classes: euploidy (EUP) and aneuploidy (ANU). ANU can be further stratified into simple aneuploidy (SA) and complex aneuploidy (CxA). Time lapse images were annotated with the developmental stage and time point at which the image was taken. Time lapse images were captured using the Embryoscope® imaging instrument.

To validate the generalizability of all trained models, a second dataset was utilized, i.e. Embryoscope+data. This dataset was also from the Center of Reproductive Medicine, but captured between 2019-2020 using a newer Embryoscope+® consisting of a total of 841 embryos (SA n=170, CxA n=261, and EUP n=410). In addition to time-lapse sequences, this dataset also contained BS, morphokinetic parameters, and maternal age for each embryo. An external dataset from IVI Valencia, the Spain dataset, was also used to validate model performances. The Spain dataset had a total of 543 embryos (SA n=309, EUP n=234) with time-lapse sequences, morphokinetic parameters, and maternal age for each embryo.

### Temporal and Spatial Processinq

Extracted time lapse image sequences were highly variable in length, frame rate, start, and end points. These variabilities resulted in numerous embryos missing information from particular time periods, lack of proper annotation which could lead to bias in model training. To mitigate these biases, a protocol was developed to clean and standardize all videos, shown below.

Standardized timepoints were designated at 30-minute intervals from 0 hpi-150 hpi (i.e. 0 hpi, 0.5 hpi, . . . 149.5 hpi, 150.0 hpi)

For each embryo, time lapse images taken closest to standardized time points are assigned to each standpoint. If there is no image close enough (within 2 hrs) to the standardized time point, a blank frame is assigned to the standardized time point. At this point, each standardized time lapse sequence has 301 frames, where each frame corresponds to a standardized test point between 0 hpi-150 hpi.

After construction of standardized time lapse sequences, frames can be extracted for video classification model development using three parameters: start hr, end hr, and interval. For example, a model trained on Day 2 embryo development would use parameters, start hr=24.0 hpi, end hr=48.0 hpi, and interval=2 hrs. This results in 13 frames.

For image classification tasks, a time point of focus can be ascertained and the frame assigned to that time point can be extracted.

By using set time points and intervals, the lengths, starting points, and ending points of all time-lapse videos can be standardized. Missing time points were imputed using nearby frames. However, some time lapse sequences were not usable for certain prediction tasks even after standardization and were therefore removed from analysis. Each frame was resized from 800×800 to 224×224. To minimize background bias in model training, a circle Hough Transform was used to segment out the embryo in each video frame. Processing was performed uniformly on Embryoscope, Embryoscope+ and Spain datasets. Video augmentation was utilized and included a random horizontal and rotations.

### General Study Architecture

For the comparison analyses, two different splits for prediction were modeled between euploidy (EUP), aneuploidy (ANU), and complex aneuploidy (CxA); these tasks were EUP vs. ANU and EUP vs. CxA. Different time point cutoffs were used to restrict the video and image within the day that the prediction model is trained for. Details of the parameters and training data for each prediction model are available upon request. Spatial features for each frame were extracted from the cleaned time lapse images of the embryos using a ImageNet pretrained VGG16 Convolutional Neural Network (CNN).

For time point and data type comparison analysis, depending on the prediction task, a selection of frame(s) was extracted from time-lapse sequences of each embryo as previously mentioned in the Temporal and Spatial Processing section. Information about selection criteria and parameters for each task are shown in Table 1. Embryos with missing clinical information were removed from the dataset.

For image classification tasks, extracted features were inputted into an eXtreme gradient boosting tree (XGBoost) model. For video classification tasks, extracted features for each frame were input into a Bidirectional Long Short-Term Memory network (BiLSTM). To address class imbalance, class weighting was implemented. Four-fold cross validation was used to evaluate performances of models. Within each fold, 25% of the training data was used for validation data, leading to a 70/30 split for training/testing. To measure model performance, accuracy, area-under-receiver-operator-curve (AUC), precision, and recall were calculated. For both the BiLSTM and XGBoost models, early-stopping was used to ensure models did not overfit on training data.

TABLE 1
Criteria and parameters for different prediction settings across time periods.

| | Video | Image | Embryoscope | Embryoscope+ |
|---|---|---|---|---|
| Time- | Classification | Classification | Data | Data |
| point | Parameters | Parameters | Distribution | Distribution |
| Day 1 | start = 6 hpi, | extracted | ANU: 414, | ANU: 169, CxA: |
| | end = 24 hpi, | timepoint = 12 | CxA: 456, | 252, EUP: 402 |
| | interval = 2 hrs | hpi | EUP: 829 | |
| Day 2 | start = 24 hpi, | extracted | ANU: 425, | ANU: 168, CxA: |
| | end = 48 hpi, | timepoint = 36 | CxA: 469, | 257, EUP: 408 |
| | interval = 2 hrs | hpi | EUP: 848 | |
| Day 3 | start = 48 hpi, | extracted | ANU: 425, | ANU: 169, CxA: |
| | end = 72 hpi, | timepoint = 60 | CxA: 467, | 261, EUP: 410 |
| | interval = 2 hrs | hpi | EUP: 847 | |
| Day 4 | start = 72 hpi, | extracted | ANU: 425, | ANU: 170, CxA: |
| | end = 96 hpi, | timepoint = 84 | CxA: 465, | 261, EUP: 410 |
| | interval = 2 hrs | hpi | EUP: 846 | |
| Day 5 | start = 96 hpi, | extracted | ANU: 415, | ANU: 170, CxA: |
| | end = 112 hpi, | timepoint = 108 | CxA: 457, | 261, EUP: 410 |
| | interval = 2 hrs | hpi | EUP: 812 | |

Start, end, and interval parameters are provided for video classification tasks. Time points at which frames were extracted for image classification are shown. Day 1 and Day 5 have relatively shorter parameter ranges (18 hrs, 16 hrs) in order to account for missing data at extremely early and late stages of development. Data distributions for Embryoscope and Embryoscope+data at different time periods are shown as well. For Embryoscope data, distributions shown are before data augmentation.

For the model-derived video-based blastocyst score and ploidy classification models, the same prediction tasks were investigated, namely EUP vs. ANU and EUP vs. CxA. Time-lapse image frames from 96 hpi-112 hpi (Day 5) were processed as per the Temporal and Spatial Processing section. The features extracted from these frames were input to a multi-task BiLSTM regression model (video regression task), which was then trained primarily to predict embryologist-derived blastocyst scores. To ensure no data leakage, the Embryoscope dataset was split 70/30 training/testing. The BiLSTM regression model was trained only using the training slice of the dataset. To capture variance in performance, four-fold cross validation was used when training the BiLSTM regression models. The predicted blastocyst score for the training split embryos from the BiLSTM regression model, along with maternal age, were used to train a logistic regression model to predict embryo ploidy. A logistic regression model was trained on each of the cross-validated BiLSTM regression models, after which the performance metrics of each of the logistic regression models were averaged. To measure model performance, accuracy, area-under-receiver-operator-curve (AUC), precision, and recall were calculated.

Significance for all experiments was determined using the Student's T-Test, adjusted for multiple testing using Bonferroni correction.

### Feature Extraction

To extract spatial features from each frame of time lapse images, an ImageNet pre-trained model from Tensorflow was utilized. A VGG16 CNN architecture was utilized to extract spatial features from images. The final layer of the pre-trained architecture performed average pooling which resulted in 512-dimensional feature vectors for each frame of each embryo.

### Comparison Prediction Models

For video classification tasks, a Bidirectional Long Short-Term Memory (BiLSTM) network was implemented. BiLSTM networks are useful for learning patterns from sequential data, which will allow it to process the temporal information from time-lapse images.¹⁶ The network architecture utilized is composed of one bidirectional LSTM layer followed by three dense layers. Architectures that utilized attention and/or multiple bidirectional LSTM layers were experimented with, but these provided no significant increases in performance. For image classification tasks, an eXtreme gradient boosting tree (XGBoost) was used. XGBoost architectures are able to learn patterns within the features extracted from each image without overfitting to the training set. Both image and video classification task models were modified to allow maternal age to be concatenated with features extracted from the pre-trained CNN. Binary cross entropy loss was used as loss functions for both BiLSTM and XGBoost models. Prediction models trained on only clinical features (BS, maternal age) used a logistic regression architecture. All models output a binary classification of either EUP or ANU/CxA (depending on the prediction task). Trained models were then validated on data from the Embryoscope+.

### MDBS Ploidy Prediction Model

For the reasons listed above, a BiLSTM architecture was used to predict blastocyst score from time-lapse sequences. The BiLSTM architecture was changed to perform multitasking, where in addition to blastocyst score, the model was trained to optionally predict expansion score, ICM score, and TE score. Multi-tasking has been used in previous studies to increase performance in scenarios where predicting different scenarios together may be advantageous to individual task performance. Similar tasks may have overlap in model weights required to come to accurate predictions, hence providing additional information for purposes of performing each task.¹⁷ Because expansion, ICM, and TE score make up the overall blastocyst score, it is reasonable to conclude that multi-tasking can be used to improve blastocyst score prediction. The BiLSTM architecture is composed of one bidirectional LSTM layer followed by two multi-unit dense layers. For each prediction task, a 1-unit dense layer is added to the model. Since all tasks of the multi-task model are regression, ‘log cosh’ was used as the loss function, and Adam was used as the optimizer. Loss weights for each prediction task within the multi-task environment were equal. Maternal age was included as a feature to the BiLSTM regression model to predict blastocyst score. Early-stopping with patience=5 was used to ensure the model was not overfitting to the training data. The second part of the model-derived video-based blastocyst score and ploidy prediction model, the logistic regression model, was fed the predicted blastocyst score, optionally in combination with maternal age, and performed a binary classification task. The logistic regression model used cross-entropy loss.

### Example 2

### Timepoint and Data Type Comparisons

A study was designed and conducted to use deep learning approaches to predict ploidy status from time-lapse sequences of embryo development. In addition, model performances were compared across time periods and between image and video classification models.

Two datasets were used in this study. The first dataset consisted of 1998 time-lapse sequences captured by the Embryoscope®. The second dataset consisted of 841 time-lapse sequences, but were captured by the Embryoscope+®. Most time lapse image sequences consisted of around 360-420 unique frames captured at intervals of 0.3 hour during 5 days of development.

PGT-A results were used as ground truth labels for all ploidy prediction tasks, with embryos being classified as euploidy (EUP) and aneuploidy (ANU); ANU was stratrified further into single aneuploidy (SA), and complex aneuploidy (CxA). SA embryos have one chromosomal abnormality (i.e. trisomy 21 or trisomy 15), whereas CxA embryos have multiple chromosomal abnormalities. The analysis focused on two prediction tasks to evaluate performances of prediction models: EUP vs ANU and EUP vs. CxA. Both datasets also included clinical information such as blastocyst score (BS) derived from morphological grade, morphokinetic parameters, and maternal age at the time of oocyte retrieval. An external dataset from IVI Valencia, Spain, was used for additional validation of certain models. In contrast to the first 2 datasets, the Spain dataset only contained EUP/ANU labels without explicit SA/CxA details and BS. Time-lapse sequence images were spatially and temporally preprocessed to reduce bias (see Methods).

Various deep learning architectures were used to predict ploidy using time lapse images and compare performances of these models across time periods and tasks. Maternal age was included as a variable in certain prediction tasks. For each task, the Embryoscope dataset was split 70/30 for training and testing, and 4-fold cross-validation was utilized. Model performance was evaluated using accuracy, AUC, precision, and recall on both Embryoscope test set and the Embryoscope+dataset (FIG. 1). A detailed description of the datasets, methodology, and different prediction tasks can be found in the preceding example.

As shown in FIG. 2, data is collected from Embryoscope and Embryoscope+data sources. Then, time-lapse images are both temporally and spatially processed to decrease bias. Horizontal and rotational augmentation is performed on time-lapse sequences. 512-dimensional features are extracted for each time-lapse image using a pretrained VGG16 architecture. Lastly, a deep learning model is trained (only on Embryoscope data) to process the frame(s), where the architecture is dependent on whether the task is one of image or video classification.

FIGS. 3A-3D depict performances of video and image classification models at different time periods on the Embryoscope test set. Each plot illustrates the performance of the prediction model in the given settings. FIGS. 3A and 3B show the image classification bar graphs and ROC plots, respectively, while FIGS. 3C and 3D show the video classification bar graphs and ROC plots, respectively. As shown in the graphs of FIGS. 3A-3D, AUCs for video-only classification tasks ranged from 0.5-0.622. AUC for image-only tasks ranged from 0.507-0.571. For both video-only and image-only classification tasks, model performance on Day 5 was significantly higher than any other day (p<0.05). AUC scores for all video-only prediction tasks on both Embryoscope and Embryoscope+dataset are shown in Table 2.

Performance on Day 1-Day 4 were not statistically different from each other for both image-only and video-only classification tasks. AUC for video+age classification tasks ranged from 0.719-0.795. AUC for image+age tasks ranged from 0.682-0.793. For both image and video classification tasks that included maternal age, there was no significant differences in performances across time periods.

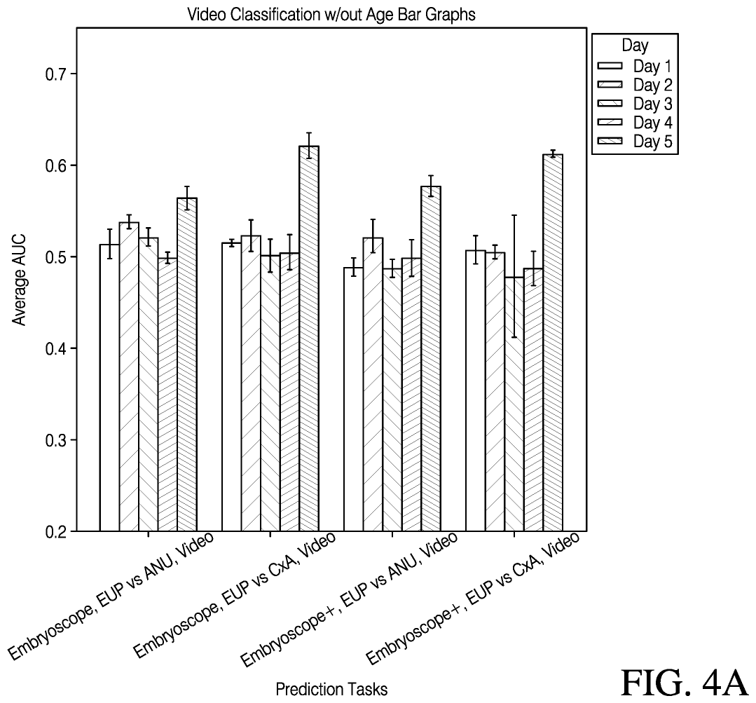

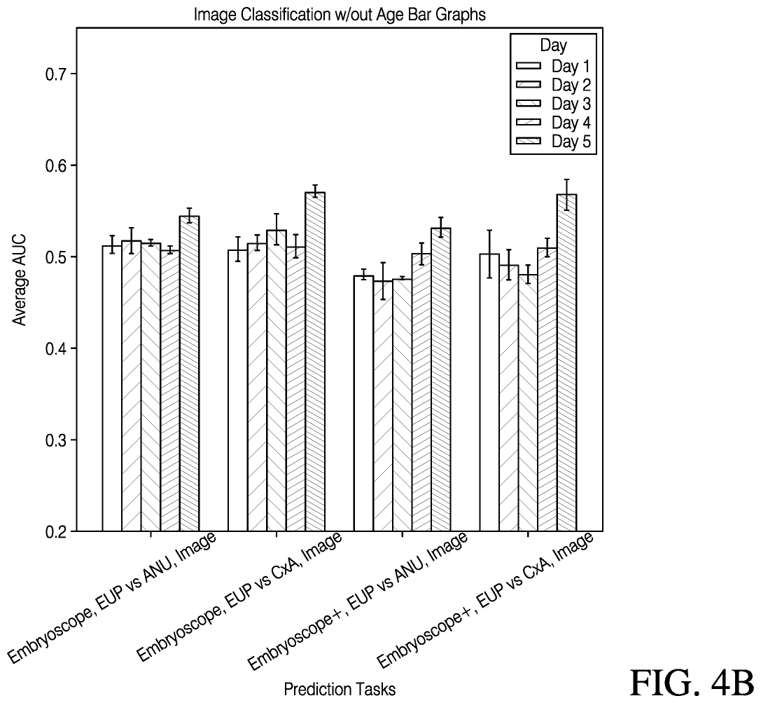

FIGS. 4A-4B show the performances across time periods of all non-age models on both the Embryoscope test set and Embryoscope+dataset. FIGS. 4A and 4B show bar graphs for the video classification and image classification, respectively, without consideration of maternal age. As shown in the graphs of FIGS. 4A-4B, for all prediction tasks, Day 5 models performed significantly better than other Day models. In both video classification and image classification tasks, there are no statistically significant differences between Embryoscope dataset performances on Day 1-4. Performances across time periods for video+age and image+age models on the Embryoscope test set and the Embryoscope+dataset are shown in Tables 3 and 4. Interestingly, there are no significant differences between time periods when maternal age is included as an input, showing that maternal age tends to diminish the returns of selecting certain time periods.

To further explore key time periods in discriminating embryo ploidy status, principal component analysis (PCA) was performed on the CNN-extracted features for all embryos within the Embryoscope dataset. Principal components (PCs) were calculated at each time point using the CNN-extracted features derived from the time-lapse frame originating at said time point. 100 principal components captured approximately 95% of the variance in the extracted features. The data points were separated by ploidy status, and the distance between ploidy status clusters was calculated at all time periods.

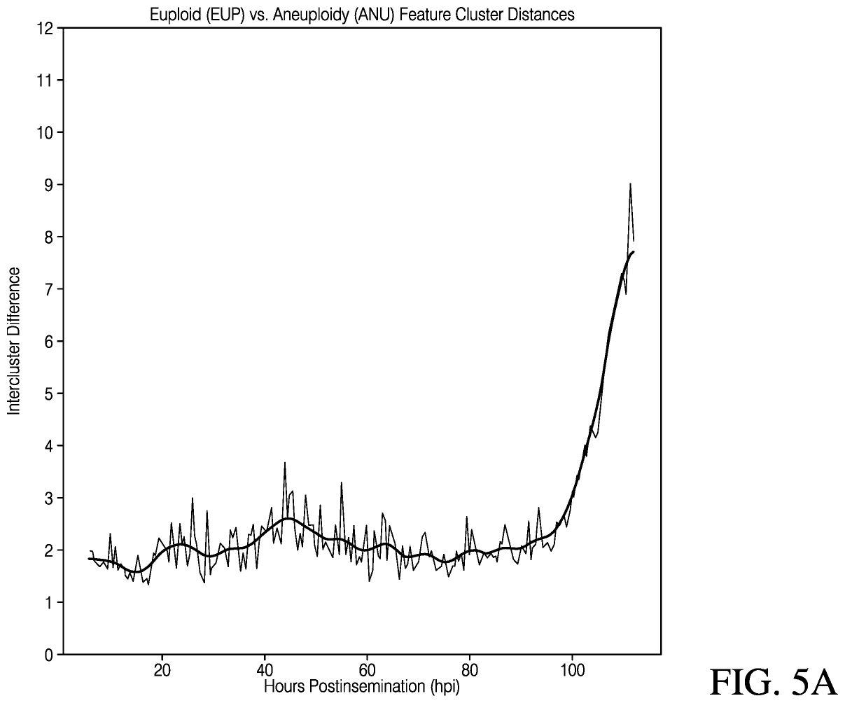

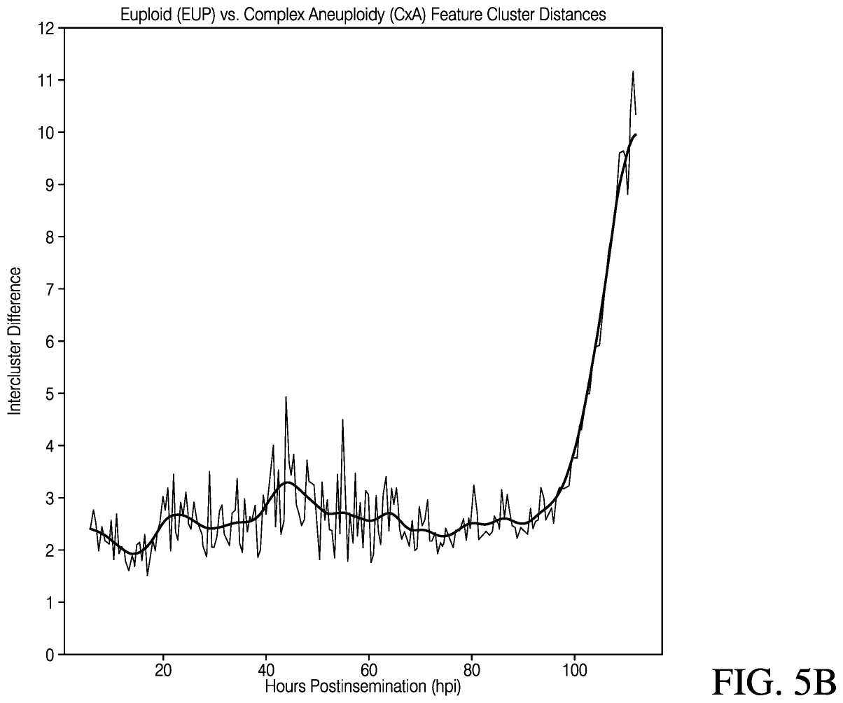

FIGS. 5A-5B show the intercluster differences at different time periods for both the EUP vs ANU (FIG. 5A) and EUP vs CxA (FIG. 5B) splits. Intercluster differences in both splits increase rapidly after 100 hpi, which occurs on Day 5. This is consistent with Day 5 performance models being significantly better at discriminating between ploidy status. Interestingly, there is a slight increase in intercluster difference between 42-46 hpi. This increase is not reflected in Day 2 model performances and may be negligent. Unsurprisingly, intercluster differences in the EUP vs. C×A split are generally higher than the EUP vs. ANU split, likely due to complex aneuploidy embryos being a more extreme case of aneuploidy. In summary, these results demonstrate that the Day 5 time period has significantly more pertinent information in predicting ploidy status compared to other time periods.

TABLE 2
Video-only classification model performance between
Day 5 and All Days, with AUC scores for
all video-only prediction tasks on both Embryoscope
and Embryoscope+ dataset. All Day models used
the following parameters: start = 6 hpi, end = 112 hpi,
interval = 2 hrs. Day 5 models perform significantly
better than All Day models on all tasks and datasets.

| Prediction | | |
|---|---|---|
| Task and | | |
| Dataset | Day 5 AUC | All Days AUC |
| EUP vs CxA, | 0.622 ± 0.014 | 0.606 ± 0.011 |
| Embryoscope | | |
| EUP vs ANU, | 0.564 ± 0.013 | 0.538 ± 0.025 |
| Embryoscope | | |
| EUP vs CxA, | 0.613 ± 0.004 | 0.589 ± 0.016 |
| Embryoscope+ | | |
| EUP vs ANU, | 0.577 ± 0.011 | 0.522 ± 0.041 |
| Embryoscope+ | | |

TABLE•3
•All•performance•metrics•for•prediction•tasks•on•Embryoscope•data,
•including•AUC, •precision, •and•recall•on•Embryoscope•
testing•data•for•all•prediction•settings.
Time•Point¤

| °¤ | ¤ | | | | | | |
|---|---|---|---|---|---|---|---|
| Discrimination• | Prediction• | ¤ | Day•1¤ | Day•2¤ | Day•3¤ | Day•4¤ | Day•5¤ |
| Task¤ | Setting¤ | Metric¤ | °¤ | °¤ | °¤ | °¤ | °¤ |
| °¤ | ¤ | AUC¤ | 0.514•± 0.016¤ | 0.538•±•0.007¤ | 0.522•±•0.01¤ | 0.499•±•0.006¤ | 0.564•± 0.013¤ |
| °¤ | Video¤ | Precision¤ | 0.546•±•0.027¤ | 0.534•± 0.013¤ | 0.542•±•0.003¤ | 0.491•±•0.049¤ | 0.585•±•0.028¤ |
| °¤ | ¤ | Recall¤ | 0.473•±•0.303¤ | 0.767•±•0.288¤ | 0.37•± 0.152¤ | 0.459•±•0.247¤ | 0.403•± 0.106¤ |
| °¤ | ¤ | AUC¤ | 0.513•± 0.01¤ | 0.518•± 0.014¤ | 0.515•± 0.004¤ | 0.507•±•0.004¤ | 0.545•±•0.008¤ |
| °¤ | Image¤ | Precision¤ | 0.527•± 0.011¤ | 0.535•± 0.012¤ | 0.532•±•0.005¤ | 0.524•±•0.008¤ | 0.547•±•0.007¤ |
| EUP•vs.•ANU¤ | ¤ | Recall¤ | 0.482•± 0.012¤ | 0.475•±•0.008¤ | 0.479•±•0.021¤ | 0.459•± 0.018¤ | 0.483•± 0.011¤ |
| °¤ | ¤ | AUC¤ | 0.719•± 0.004¤ | 0.732•±•0.004¤ | 0.724•±•0.009¤ | 0.726•±•0.009¤ | 0.731•±•0.006¤ |
| °¤ | Video•+• | Precision¤ | 0.673•±•0.008¤ | 0.696•±•0.008¤ | 0.676•± 0.012¤ | 0.669•± 0.021¤ | 0.699•± 0.013¤ |
| | Age¤ | | | | | | |
| °¤ | ¤ | Recall¤ | 0.722•±•0.039¤ | 0.675•±•0.031¤ | 0.692•±•0.027¤ | 0.717•± 0.047¤ | 0.631•±•0.032¤ |
| °¤ | ¤ | AUC¤ | 0.682•±•0.014¤ | 0.702•±•0.004¤ | 0.689•±•0.006¤ | 0.688•±•0.011¤ | 0.705•±•0.004¤ |
| °¤ | Image•+• | Precision¤ | 0.657•± 0.014¤ | 0.669•±•0.012¤ | 0.651•±•0.005¤ | 0.664•±•0.01¤ | 0.677•±•0.003¤ |
| | Age¤ | | | | | | |
| °¤ | ¤ | Recall¤ | 0.624•±•0.01¤ | 0.655•±•0.009¤ | 0.643•±•0.01¤ | 0.639•±•0.008¤ | 0.639•±•0.005¤ |
| °¤ | ¤ | AUC¤ | 0.515•± 0.004¤ | 0.523•±•0.018¤ | 0.501•± 0.018¤ | 0.505•± 0.019¤ | 0.622•± 0.014¤ |
| °¤ | Video¤ | Precision¤ | 0.689•±•0.007¤ | 0.687•±•0.012¤ | 0.673•±•0.006¤ | 0.641•±•0.078¤ | 0.805•±•0.043¤ |
| °¤ | ¤ | Recall¤ | 0.518•± 0.212¤ | 0.65•± 0.159¤ | 0.556•± 0.175¤ | 0.496•±•0.323¤ | 0.41•± 0.149¤ |
| °¤ | ¤ | AUC¤ | 0.508•± 0.013¤ | 0.515•± 0.009¤ | 0.529•± 0.017¤ | 0.511•± 0.013• | 0.571•±•0.007¤ |
| °¤ | Image¤ | Precision¤ | 0.684•±•0.006¤ | 0.69•±•0.002¤ | 0.691•±•0.003¤ | 0.692•±•0.004¤ | 0.696•±•0.002¤ |
| EUP•vs.•C × A¤ | ¤ | Recall¤ | 0.845•±•0.009¤ | 0.832•±•0.008¤ | 0.851•±•0.007¤ | 0.835•±•0.001¤ | 0.807•±•0.008¤ |
| °¤ | ¤ | AUC¤ | 0.772•±•0.008¤ | 0.781•±•0.008¤ | 0.778•± 0.013¤ | 0.782•±•0.008¤ | 0.795•±•0.006¤ |
| °¤ | Video•+• | Precision¤ | 0.836•± 0.017¤ | 0.849•±•0.008¤ | 0.855•± 0.011¤ | 0.852•± 0.016¤ | 0.854•± 0.015¤ |
| | Age¤ | | | | | | |
| °¤ | ¤ | Recall¤ | 0.739•±•0.029¤ | 0.718•±•0.032¤ | 0.712•± 0.022¤ | 0.711•± 0.037¤ | 0.71•±•0.061¤ |
| °¤ | ¤ | AUC¤ | 0.775•±•0.002¤ | 0.793•±•0.007¤ | 0.764•±•0.003¤ | 0.777•±•0.009¤ | 0.784•±•0.006¤ |
| °¤ | Image•+• | Precision¤ | 0.802•±•0.008¤ | 0.809•±•0.005¤ | 0.797•±•0.007¤ | 0.811•±•0.005¤ | 0.808•±•0.005¤ |
| | Age¤ | | | | | | |
| °¤ | °¤ | Recall¤ | 0.843•±•0.01¤ | 0.842•±•0.008¤ | 0.827•±•0.01¤ | 0.843•±•0.009¤ | 0.834•±•0.006¤ |

TABLE•4
•All•performance•metrics•for•prediction•tasks•on•Embryoscope•+•data,
•including•AUC, •precision, •and•recall•on•
Embryoscope•+•data•for•all•prediction•settings.
Time•Point¤

| °¤ | ¤ | | | | | | |
|---|---|---|---|---|---|---|---|
| Discrimination• | Prediction• | ¤ | Day•1¤ | Day•2¤ | Day•3¤ | Day•4¤ | Day•5¤ |
| Task¤ | Setting¤ | Metric¤ | °¤ | °¤ | °¤ | °¤ | °¤ |
| °¤ | °¤ | AUC¤ | 0.489•±•0.01¤ | 0.522•±•0.018¤ | 0.487•±•0.01¤ | 0.499•±•0.02¤ | 0.577•±•0.011¤ |
| °¤ | Video¤ | Precision¤ | 0.356•±•0.206¤ | 0.503•±•0014¤ | 0.101•±•0.176¤ | 0•355•±•0.205¤ | 0.519 ± 0017¤ |
| °¤ | ¤ | Recall¤ | 0.379•±•0.369¤ | 0.83•±•0.213¤ | 0.018•±•0.032¤ | 0.645•±•0.399¤ | 0.709•±•0.076¤ |
| °¤ | ¤ | AUC¤ | 0.48•±•0.006¤ | 0.473•±•0.02¤ | 0.476•±•0.002¤ | 0•503•±•0.012¤ | 0.532•±•0.011¤ |
| °¤ | Image¤ | Precision¤ | 0.471•±•0.011¤ | 0.392•±•0.07¤ | 0.432•±•0.011¤ | 0.509•±•0.014¤ | 0.5•±•0.006¤ |
| EUP•vs.•ANU¤ | ¤ | Recall¤ | 0.504•±•0.038¤ | 0.074•±•0.022¤ | 0.175•±•0.036¤ | 0.26•±•0.033¤ | 0.774•±•0.015¤ |
| | | AUC¤ | 0.695•±•0.008¤ | 0.704•±•0.003¤ | 0.696•±•0.006¤ | 0.698•±•0.007¤ | 0.684•±•0.014¤ |
| °¤ | Video•+• | Precision¤ | 0.639•±•0.016¤ | 0.634•±•0•005¤ | 0.639•±•0.013¤ | 0.607•±•0.013¤ | 0.596•±•0.016¤ |
| | Age¤ | | | | | | |
| °¤ | ¤ | Recall¤ | 0.707•±•0.055¤ | 0.722•±•0.013¤ | 0.663•±•0.048¤ | 0.762•±•0.054¤ | 0.75•±•0.03¤ |
| °¤ | ¤ | AUC¤ | 0.669•±•0.007¤ | 0.658•±•0.01¤ | 0.664•±•0.012¤ | 0.665•±•0.012¤ | 0.667•±•0.012¤ |
| °¤ | Image•+• | Precision¤ | 0.61•±•0.011¤ | 0.625•±•0.018¤ | 0.632•±•0.006¤ | 0.629•±•0.014¤ | 0.572•±•0.003¤ |
| | Age¤ | | | | | | |
| °¤ | ¤ | Recall¤ | 0.702•±•0.025¤ | 0•388•±•0.076¤ | 0.53•±•0•036¤ | 0.491•±•0.067¤ | 0.821•±•0.015¤ |
| °¤ | ¤ | AUC¤ | 0.508•±•0.015¤ | 0.505•±•0•008¤ | 0.479•±•0.067¤ | 0.487•±•0.019¤ | 0.613•±•0004¤ |
| °¤ | Video¤ | Precision¤ | 0.712•±•0.167¤ | 0.618•±•0004¤ | 0.572•±•0.094¤ | 0.616•±•0.011¤ | 0.658•±•0.015¤ |
| °¤ | ¤ | Recall¤ | 0.365•±•0.323¤ | 0.92•±•0.067¤ | 0.209•±•0.176¤ | 0.53•±•0.44¤ | 0.725•±•0•041¤ |
| °¤ | ¤ | AUC¤ | 0.503•±•0.026¤ | 0.491•±•0.017¤ | 0.481•±•0.01¤ | 0.51•±•0.01¤ | 0.568•±•0.017¤ |
| °¤ | Image¤ | Precision¤ | 0.612•±•0.007¤ | 0.591•±•0.013¤ | 0.595•±•0.015¤ | 0.614•±•0.01¤ | 0.613•±•0.004¤ |
| EUP•vs.•C + A¤ | ¤ | Recall¤ | 0.89•±•0.029¤ | 0.241•±•0.014¤ | 0.421•±•0.025¤ | 0.719•±•0.126¤ | 0.958•±•0.012¤ |
| | | AUC¤ | 0.747•±•0.015¤ | 0.742•±•0.006¤ | 0.732•±•0.013¤ | 0.735•±•0.016¤ | 0.741•±•0.006¤ |
| °¤ | Video•+• | Precision¤ | 0.758•±•0.015¤ | 0.767•1•0•008¤ | 0.77•±•0.012¤ | 0.745•±•0.017¤ | 0.723•±•0.02¤ |
| | Age¤ | | | | | | |
| °¤ | ¤ | Recall¤ | 0.785•±•0.037¤ | 0.753•±•0.013¤ | 0.662•±•0.113¤ | 0.78•±•0.028¤ | 0.826•±•0.068¤ |
| °¤ | ¤ | AUC¤ | 0.738•±•0.004¤ | 0.739•±•0.006¤ | 0.721•±•0.007¤ | 0.73•±•0.008¤ | 0.732•±•0.009¤ |
| °¤ | Image•+• | Precision¤ | 0.707•±•0.006¤ | 0.772•±•0.008¤ | 0.769•±•0.011¤ | 0.733•±•0.02¤ | 0.676•±•0.017¤ |
| | Age¤ | | | | | | |
| °¤ | °¤ | Recall¤ | 0.86•±•0.022¤ | 0.758•±•0.009¤ | 0.726•±•0.015¤ | 0.811•±•0.032¤ | 0.926•±•0.022¤ |

### Example 3

### Image and Video Comparison Analysis

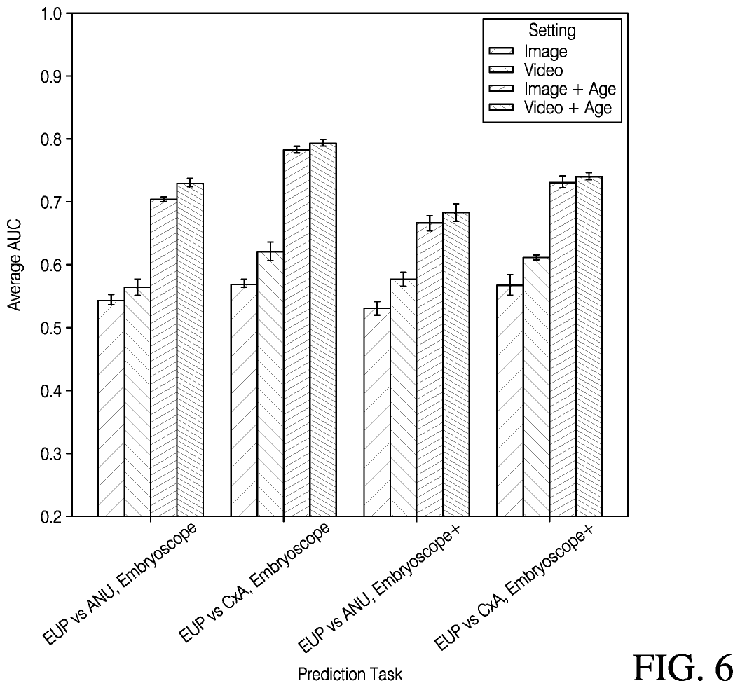

FIG. 6 shows performances of all Day 5 models on both Embryoscope and Embryoscope+datasets. Performances for both Embryoscope and Embryoscope+datasets for all prediction tasks are depicted. Average AUC and standard errors are shown for each prediction setting.

Day 5 was chosen as the main time point of image/video comparison because earlier analysis showed that Day 5 provided non-negligible information about ploidy discrimination. In the Embryoscope dataset, video classification models (video-only and video+age) performed significantly better than image classification models at all prediction tasks (p<0.05). Video-only models performed significantly better than image-only models at all tasks on Embryoscope+data as well (p<0.05). Video+age and image+age models performed comparably on Embryoscope+data. Comparisons of image and video classification models on Days 1-4 can be derived from Tables 3 and 4. Comparisons of Day 5 model architectures are shown in Table 5. No significant differences were noted between video-only and age-only models at earlier time periods. Video+age models performed significantly better than image+age models on both Embryoscope and Embryoscope+datasets across Day 1-4.

Video-only and image-only models perform significantly (p<0.05) worse than models trained only on blastocyst score (BS) or maternal age (FIG. 7A). The blastocyst score is a quality metric based on the conversion of blastocyst embryologist-annotated morphological grades on Day 5. Time to blastulation (tB) only models perform comparably with video-only classification models on the Embryoscope dataset. While video+age and image+age models perform significantly better than age-only models on the Embryoscope dataset, they perform significantly worse on Embryoscope+data (FIG. 7B). In summary, video-based models were found to perform significantly better than single-image based models, most likely due to the added temporal information.

TABLE 5
Day 5 Video Classification Model Architecture
Comparisons. AUC performances on the
Embryoscope dataset with BiLSTM and XGBoost
video classification Day 5 models. Frame features for
each embryo were concatenated before being inputted
into XGBoost models. BiLSTM models perform
significantly better than XGBoost models.

| | BiLSTM AUC | XGBoost AUC |
|---|---|---|
| EUP vs CxA, | 0.622 ± 0.014 | 0.58 ± 0.02 |
| Video | | |
| EUP vs ANU, | 0.564 ± 0.013 | 0.546 ± 0.009 |
| Video | | |

TABLE 6
Performance comparison of two Day 5 Image
Classification models. AUC performances on the
Embryoscope test set for Day 5 image classification
models. The left column uses single images from
108 hpi, and the right column uses single images
from 112 hpi. Differences in performance are non-
significant for all prediction tasks and settings.

| Prediction | | |
|---|---|---|
| Task | 108 hpi | 112 hpi |
| and Setting | AUC | AUC |
| EUP vs CxA, | 0.571 ± 0.007 | 0.57 ± 0.016 |
| Image | | |
| EUP vs ANU, | 0.545 ± 0.008 | 0.552 ± 0.014 |
| Image | | |
| EUP vs CxA, | 0.784 ± 0.006 | 0.782 ± 0.009 |
| Image + Age | | |
| EUP vs ANU, | 0.705 ± 0.004 | 0.706 ± 0.007 |
| Image + Age | | |

### Example 4

### Ploidy Prediction Model with Model-Derived Blastocyst Score

In addition to performing comparisons between time periods and data types, a new fully automated model for ploidy prediction is presented, namely the model-derived video-based blastocyst score and ploidy classification/prediction model (FIG. 8). As shown in FIG. 8, features are extracted from time-lapse image frames as shown in FIG. 2, steps 1-5. These features are fed into a multitask BiLSTM model (1) which is trained to predict blastocyst score as well as other embryologist-annotated morphological scores. Predicted blastocyst scores are inputted into a logistic regression model (2) to perform ploidy classification.

This model consists of two steps. The first part is blastocyst score prediction from processed Day 5 time-lapse video input. In the second step, the predicted BS (or MDBS) is used to predict ploidy status of the embryo. For this second step, architectures were developed which had the capacity to include maternal age as an input feature. Performance of the model-derived video-based blastocyst score and ploidy classification/prediction model was evaluated on both EUP vs CxA and EUP vs ANU splits. Model performance was evaluated using accuracy, AUC, precision, and recall on both Embryoscope test set and Embryoscope+dataset.

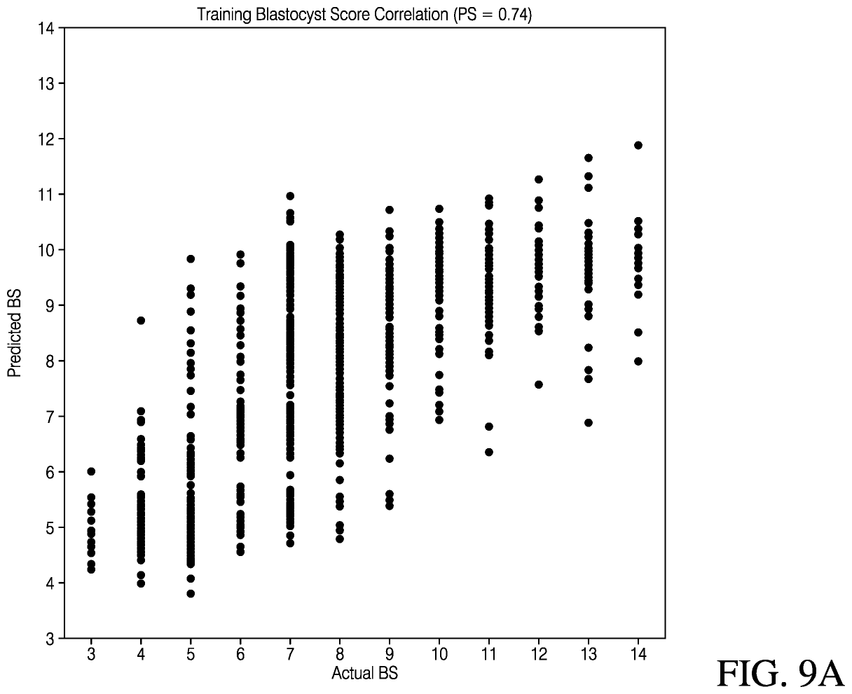

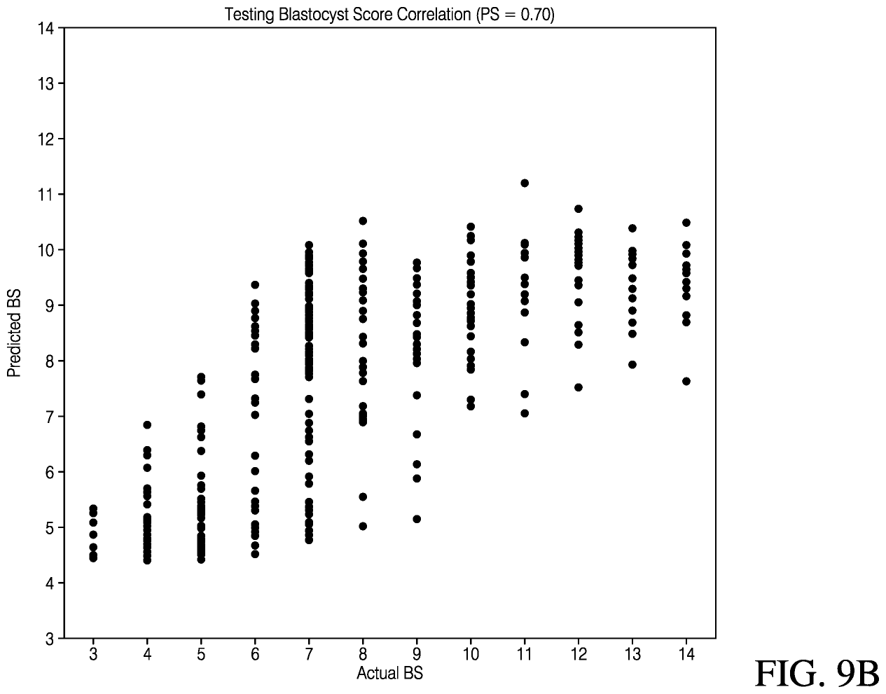

The first part of the model-derived video-based blastocyst score ploidy model is blastocyst score prediction. FIGS. 9A-9B show Pearson correlations between model-derived blastocyst scores (MDBS) and manual blastocyst scores (BS) (ground-truth) for the Embryoscope training and test sets (FIG. 9A and FIG. 9B, respectively). Correlation between scores is ˜0.7 for both datasets, indicating moderate correlation strength between MDBS and manual BS scores.

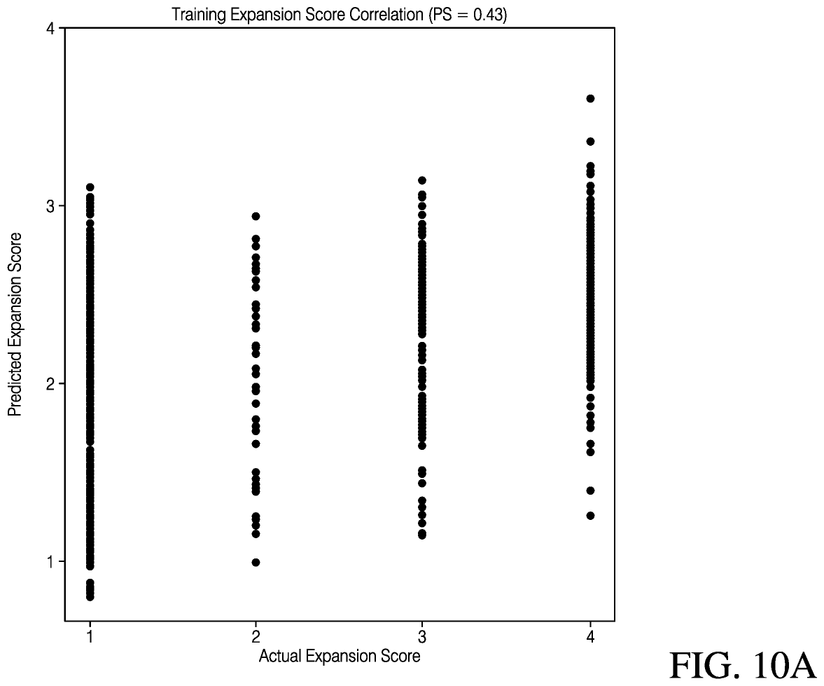

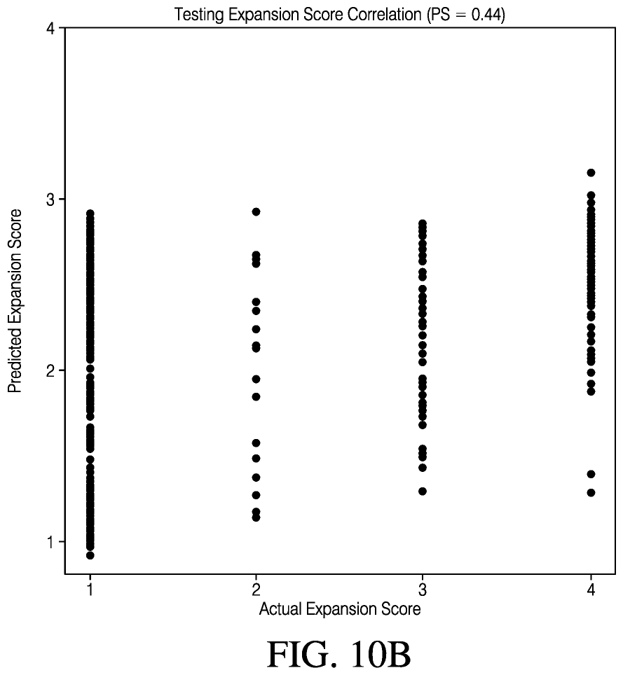

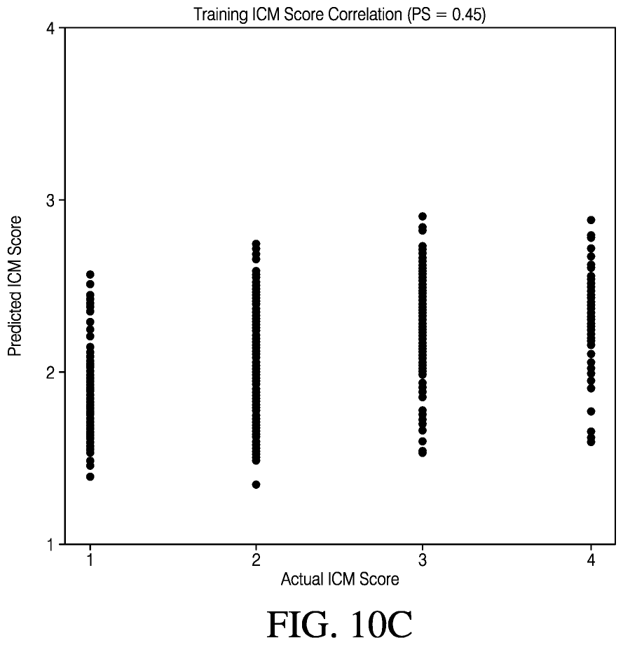

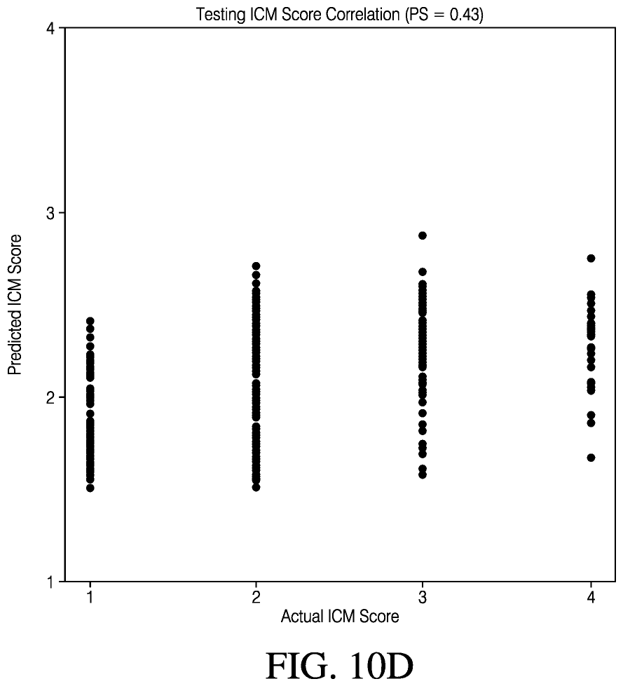

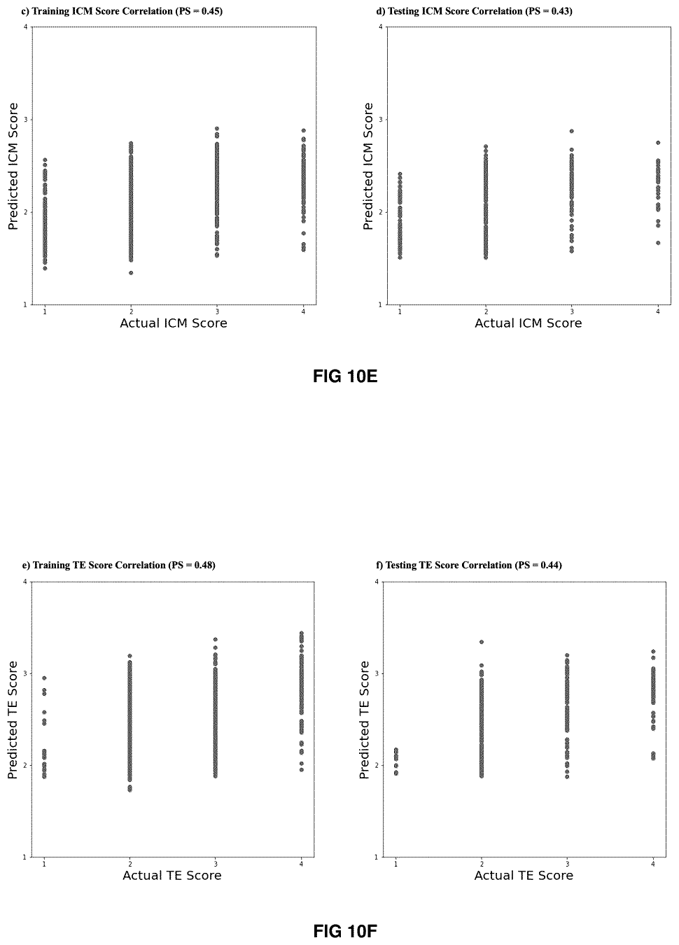

FIGS. 10A-10F show Pearson correlations between predicted and acutal multitask scores, including expansion scores for the Embryoscope training and test sets (FIG. 10A and FIG. 10B, respectively), ICM scores for the Embryoscope training and test sets (FIG. 10C and FIG. 10D, respectively), and TE scores for the Embryoscope training and test sets (FIG. 10E and FIG. 10F, respectively). As shown in the graphs of FIGS. 10A-10F, Pearson correlations between predicted and actual scores for other embryologist metrics are moderately strong as well. The second part of the model-derived video-based blastocyst score and ploidy model is ploidy classification. On the Embryoscope test set, the model-derived video-based blastocyst score ploidy model trained to discriminate EUP vs ANU had an AUC of 0.66±0.008, which increased to 0.76±0.002 when maternal age was included in ploidy prediction. For the EUP vs CxA task, the model had an AUC of 0.708±0.006 which increased to 0.827±0.004 when maternal age was included. Detailed performances of the MDBS-Ploidy models are shown in Table 7.

TABLE 7
All performance metrics for MDBS models on all prediction tasks,
settings, and test sets, including AUC, precision, and recall.

| | Prediction | Prediction | | | |
|---|---|---|---|---|---|
| Test Set | Task | Setting | AUC | Recall | Precision |
| Embryoscope | EUP vs. CxA | MDBS | 0.708 ± 0.006 | 0.591 ± 0.019 | 0.797 ± 0.019 |
| | | MDBS + Age | 0.827 ± 0.004 | 0.745 ± 0.016 | 0.878 ± 0.008 |
| | EUP vs. ANU | MDBS | 0.659 ± 0.008 | 0.546 ± 0.009 | 0.643 ± 0.027 |
| | | MDBS + Age | 0.76 ± 0.002 | 0.688 ± 0.01 | 0.705 ± 0.005 |
| Embryoscope+ | EUP vs. CxA | MDBS | 0.67 ± 0.001 | 0.848 ± 0.013 | 0.668 ± 0.001 |
| | | MDBS + Age | 0.771 ± 0.002 | 0.879 ± 0.004 | 0.722 ± 0.006 |
| | EUP vs. ANU | MDBS | 0.626 ± 0.001 | 0.813 ± 0.027 | 0.536 ± 0.001 |
| | | MDBS + Age | 0.717 ± 0.002 | 0.85 ± 0.003 | 0.594 ± 0.004 |

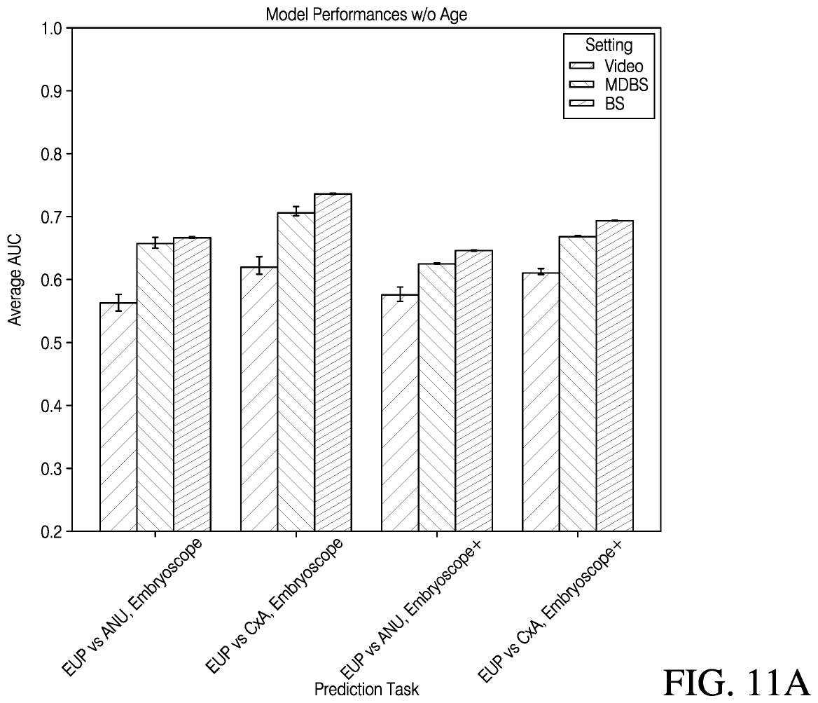

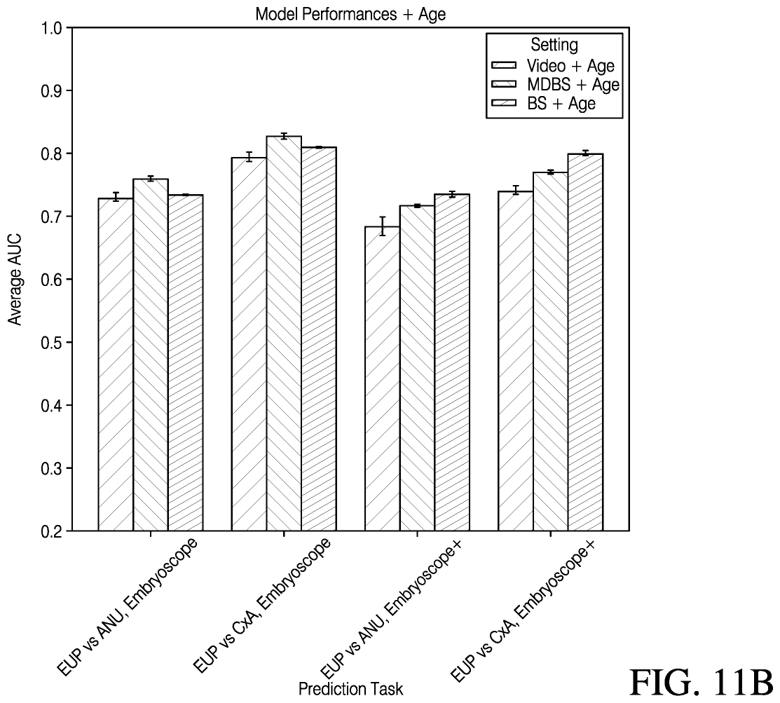

FIGS. 11A-11B show the performance of the MDBS-Ploidy model (in middle) compared to the Day 5 Video model (on left) and embryologist-annotated blastocyst score model (on right). In FIGS. 11A-11B, mean AUC scores and standard deviation for Day 5 video, MDBS, and embryologist-annotated BS trained models are shown, with performances shown on both the Embryoscope and Embryoscope+dataset for both prediction tasks. Performances of models without maternal age (FIG. 11A) and with maternal age (FIG. 11B) are shown.

In all prediction settings (with or without age), test sets, and prediction tasks (EUP vs ANU and EUP vs CxA) the MDBS-Ploidy model outperforms the Day 5 Video model (p<0.05). In the prediction setting where maternal age is not included in ploidy prediction, the embryologist-annotated BS model outperforms (p<0.05) the MDBS model in all prediction tasks except for EUP vs ANU on the Embryoscope test set (FIG. 11A). In the prediction setting where maternal age is included, the MDBS model outperforms the embryologist-annotated blastocyst score model in the Embryoscope test set (p<0.05). However, the MDBS model underperforms in comparison to the embryologist-annotated blastocyst score model on the Embryoscope+dataset.

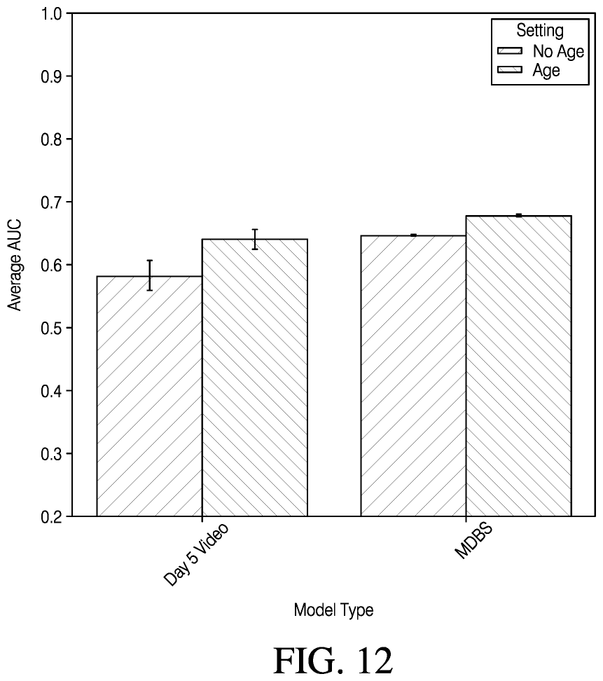

The performance of the MDBS model was also compared with the Day 5 Video model on the Spain dataset (FIG. 12). In FIG. 12, average AUC with standard errors are shown for all EUP vs ANU prediction tasks on the Spain dataset for both Day 5 Video and MDBS-Ploidy models. The darker (left) bars depict model performances where maternal age was not included in the ploidy model as an additional feature, whereas the lighter (right) bars depict performances where maternal age was added.

The Spain dataset contains embryos labeled either as ANU or EUP, hence the only task we could measure model performance on is EUP vs ANU. The MDBS model significantly outperforms the Day 5 video model both with and without the addition of maternal age (p<0.05). Unlike the embryos observed at Weill Cornell Medicine (Embryoscope and Embryoscope+datasets), the embryos from Spain are induced to hatch on Day 3 which could affect embryo development Day 4+. This means that embryos within the Spain dataset have the potential to have several different characteristics after Day 4 as compared to the embryos at Weill Cornell Medicine. Despite this, we see that model performance (without maternal age) is comparable to model performance on Weill Cornell Medicine datasets. This may indicate the generalizability of our model to multiple clinics, even those with practices not accounted for within the training data. However, when maternal age is included, performances on the Spain dataset are significantly lower than performances on Weill Cornell Medical datasets. This can be due to the differing age distributions at each clinic. Unlike Spain where IVF is more affordable and accessible due to various healthcare insurance policies, in the United States, IVF can be an expensive procedure limiting its accessibility to those with the financial means to undergo the procedure.^{12,13} This likely results in the differing maternal age distributions we see within the datasets.

### Example 5

### MDBS Ploidy Model API for Clinical Use

A web-based application programming interface (API) for the MDBS ploidy model can be utilized to perform a classification task, in accordance with the preceding examples and various embodiments described herein. The platform utilizes video of a blastocyst from Day 5 (96 hpi-112 hpi), where the video includes at least two time-lapse image frames. Users then have the option to include patient age, and optionally further detail regarding morphological assessment, and morphokinetic parameters. The output results include probabilities for each classifier.

The API is a user-friendly web interface that allows embryologists and clinicians to quickly predict embryo ploidy by utilizing video of embryos at Day 5 (96 hpi-112 hpi), and inputting additional features, such as, for example, maternal age at the time of oocyte retrieval, and optionally further detail regarding morphological assessment (blastocyst score or blastocyst grade), and morphonkinetic parameters (tPnF to tSB) as inputs. At a minimum, an image is required; the other inputs are optional, with the inclusion of maternal age being the most favorable.

After inputting the image and/or various clinical parameters, the interface reports probabilities for each of the following classification tasks: Aneuploid vs. Euploid, and Complex Aneuploid vs. Euploid. The back-end of the platform recognizes the inputs being included by the user and selects the appropriately trained model to be used. Each of the classification tasks has its own unique model weights to create predictions (probabilities).

### VIII. Additional Considerations

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the disclosure.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

The various methods and techniques described above provide a number of ways to carry out the disclosure. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the disclosure extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, now their specific configuration, communication and physical orientation with respect to each other. One skilled in the art should readily appreciate that these components can have various configurations and physical orientations (e.g., wholly separate components, units and subunits of groups of components, different communication regimes between components).

In closing, it is to be understood that the embodiments of the application disclosed herein are illustrative of the principles of the embodiments of the disclosure. Although specific embodiments and applications of the disclosure have been described in this specification, these embodiments and applications are exemplary only, and many variations are possible. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### IX. References

All references cited herein are incorporated by reference in their entirety. Also incorporated herein by reference in their entirety, and for all purposes, include: U.S. Provisional Application No. 63/308,710, INTEGRATED FRAMEWORK FOR HUMAN EMBRYO PLOIDY PREDICTION USING ARTIFICIAL INTELLIGENCE, filed on Feb. 10, 2022; U.S. Provisional Application No. 63/433,197, INTEGRATED FRAMEWORK FOR HUMAN EMBRYO PLOIDY PREDICTION USING ARTIFICIAL INTELLIGENCE, filed on Dec. 16, 2022; and International Patent Application No. PCT/US2023/062368, INTEGRATED FRAMEWORK FOR HUMAN EMBRYO PLOIDY PREDICTION USING ARTIFICIAL INTELLIGENCE, filed on Feb. 10, 2023.

Additional references mentioned in the disclosure include:
1. Ma, R. C. W., Ng, N. Y. H., Cheung, L. P. Assisted Reproduction Technology and long-term Cardiometabolic Health in The offspring. PLOS Medicine. (2021)
2. Niakan K, Han J, Pedersen R, et al. Human pre-implantation embryo development. Development (Cambridge, England), 139(5), 829-841. (2012)
3. Niederberger, Craig et al. Forty years of IVF. Fertility and sterility vol. 110. (2018).
4. Greco, E., Litwicka, K., Minasi, M. G., Cursio, E., Greco, P. F., & Barillari, P. Preimplantation Genetic Testing: Where We Are Today. International journal of molecular sciences, 21(12), 4381. (2020).
5. Zhang, Y. X., Chen, J. J., Nabu, S., Yeung, Q., Li, Y., Tan, J. H., Suksalak, W., Chanchamroen, S., Quangkananurug, W., Wong, P. S., Chung, J., & Choy, K. W. The Pregnancy Outcome of Mosaic Embryo Transfer: A Prospective Multicenter Study and Meta-Analysis. Genes, 11(9), 973. (2020).
6. Khosravi, P., Kazemi, E., Zhan, Q. et al. Deep learning enables robust assessment and selection of human blastocysts after in vitro fertilization. Npj Digit. Med. 2, 21 (2019).
7. Barnes, J. et al. Noninvasive detection of blastocyst ploidy (Euploid vs. aneuploid) using artificial intelligence (AI) with Deep Learning Methods. Fertility and Sterility 114, (2020).
8. Silver, D. H. Feder, M. Gold-Zamir, Y. Polsky, A. L. Rosentraub, S. Shachor, E. Weinberger, A. Mazur, P. Zukin, V. D. Bronstein, A. M. Data-Driven Prediction of Embryo Implantation Probability Using IVF Time-lapse Imaging. arXiv. (2020).
9. Lee, C.-I. et al. End-to-end deep learning for recognition of ploidy status using time-lapse videos. Journal of Assisted Reproduction and Genetics 38, 1655-1663 (2021).
10. Campbell, A. et al. Modelling a risk classification of aneuploidy in human embryos using non-invasive morphokinetics. Reproductive BioMedicine Online 26, 477-485 (2013).
11. Gardner, D. K., Balaban, B. Assessment of human embryo development using morphological criteria in an era of time-lapse, algorithms and ‘OMICS’: Is looking good still important? Molecular Human Reproduction 22, 704-718 (2016).
12. Pierce, N., Mocanu, E. Female age and assisted Reproductive Technology. Global Reproductive Health 3, (2018).
13. Alon, I., Dominguez, J. P. Assisted reproduction in Spain, outcome and socioeconomic determinants of access. (2021).
14. Minasi, M. G. et al. Correlation between aneuploidy, standard morphology evaluation and morphokinetic development in 1730 biopsied blastocysts: A consecutive case series study. Human Reproduction 31, 2245-2254 (2016).
15. Zhan, Q. et al. Blastocyst score, a blastocyst quality ranking tool, is a predictor of blastocyst ploidy and implantation potential. F&S Reports 1, 133-141 (2020).
16. Yousaf, K., Nawaz, T. A deep learning-based approach for inappropriate content detection and classification of YouTube videos. IEEE Access 10, 16283-16298 (2022).
17. Caruana, R. Multitask Learning. Machine Learning 28, 41-75. (1997).

1. A non-invasive method of predicting ploidy status of an embryo, the method comprising:
receiving a dataset comprising video comprising a plurality of image frames of the embryo;
analyzing the dataset by one or more machine and/or deep learning model via one or more classification task applied to the dataset; and
generating an output prediction of the ploidy status of the embryo.

2. The method of claim 1, wherein the prediction of the ploidy status of the embryo comprises a probability.

3. (canceled)

4. The method of claim 1, wherein the classification task is a binary classification task which provides a probability for the embryo of being euploid vs. aneuploid; or euploid vs complex aneuploid.

5.-7. (canceled)

8. The method of claim 1, the method further comprising acquiring the plurality of image frames.

9. The method of claim 1, wherein the plurality of image frames are acquired via time-lapse microscopy.

10. The method of claim 1, wherein the plurality of image frames are captured at Day 5 of embryo development, or wherein each image of the plurality of image frames is captured from 96-112 hours post insemination (hpi).

11. (canceled)

12. The method of claim 1, wherein the plurality of image frames comprises one, two, three, four, five, or more image frames captured per hour for two or more consecutive or non-consecutive hours during Day 5 of embryo development.

13-14. (canceled)

15. The method of claim 1, wherein the model further generates an output comprising one or more clinical and/or morphological feature scores for the embryo.

16. The method of claim 15, wherein the one or more clinical and/or morphological feature scores for the embryo comprises blastocyst score (BS), expansion score (ES), inner-cell mass (ICM) score, and/or trophectoderm (TE) score.

17. (canceled)

18. The method of claim 1, wherein the dataset further comprises one or more clinical and/or morphological features for the embryo.

19. The method of claim 18, wherein the one or more clinical features for the embryo comprise maternal age at the time of oocyte retrieval.

20. The method of claim 19, wherein the one or more clinical and/or morphological features for the embryo comprise one or more morphokinetic parameters/annotations, one or more blastocyst morphological assessments, and/or preimplantation genetic testing for aneuploidy (PGT-A).

21. The method of claim 20, wherein the blastocyst morphological assessments comprise blastocyst grade (BG), blastocyst score (BS), time to blastocyst (tB), and/or artificial intelligence-driven predicted blastocyst score (AIBS).

22. The method of claim 21, wherein the BS score determination comprises converting inner cell mass (ICM), trophectoderm (TE), and/or expansion grades into numerical values, and additionally comprises an input based on day of blastocyst formation; and/or wherein BG determination comprises using a grading system comprising assessments of ICM, TE, and/or expansion.

23. (canceled)

24. The method of claim 20, wherein the morphokinetic parameters comprise time of pro-nuclear fading (tPnF), time to 2 cells (t2), time to 3 cells (t3), time to 4 cells (t4), time to 5 cells (t5), time to 6 cells (t6), time to 7 cells (t7), time to 8 cells (t8), time to 9 cells (t9), time of morula (tM), and/or time of the start of blastulation (tSB).

25.-26. (canceled)

27. The method of claim 18, wherein maternal age and/or blastocyst score (BS) are weighted more heavily than other clinical features based on one or more classification task.

28. The method of claim 27, wherein the clinical and/or morphological features are weighted in order of maternal age at the time of oocyte retrieval, blastocyst grade and/or blastocycst score, and/or morphokinetic parameters.

29. The method of claim 28, wherein blastocyst score correlates positively, and/or wherein maternal age correlates negatively with embryo ploidy status.

30. (canceled)

31. The method of claim 1, the method further comprising pre-processing the dataset prior to analysis.

32. The method of claim 31, wherein pre-processing the dataset comprises removing faulty image frames and/or imputing values for any missing image frames via median imputation.

33. (canceled)

34. The method of claim 1, wherein the output prediction is determined based on machine and/or deep learning and regression analysis.

35. The method of claim 1, wherein the analysis comprises regression analysis; and/or wherein the analysis comprises determination of an artificial intelligence-driven predicted blastocyst score (AIBS) for the embryo.

36. The method of claim 35, wherein the regression analysis comprises a LASSO regression and/or logistic regression applied to the plurality of image frames and/or one or more clinical and/or morphological features.

37. (canceled)

38. The method of claim 1, wherein the image frames and/or clinical features are combined and analyzed by machine and/or deep learning in two fully-connected layers; and/or wherein the machine learning comprises a convolutional neural network (CNN) and/or a Bidirectional Long Short-Term Memory (BiLSTM) network.

39.-41. (canceled)

42. The method of claim 1, the method further comprising:
training the one or more machine learning model using training data,
wherein the training data comprises a plurality of probabilities, and/or model- or embryologist-derived or provided clinical features for a plurality of subjects and a plurality of embryo ploidy statuses for the plurality of subjects.

43. The method of claim 1, the method further comprising predicting embryo viability based on the embryo ploidy status, wherein an embryo having a stronger probability of being euploid has a higher probability of being viable.

44. The method of claim 1, wherein the method is used for improving embryo selection for implantation during in vitro fertilization; and/or wherein the method is used for selecting and/or prioritizing an embryo for preimplantation genetic testing for aneuploidy (PGT-A) biopsy and/or implantation during in vitro fertilization; and/or wherein the method is used in combination with traditional methods of embryo selection and prioritization for implantation and/or recommendation for PGT-A during in vitro fertilization.

45.-46. (canceled)

47. The method of claim 1, the method further comprising improving an outcome in a subject undergoing in vitro fertilization, wherein an embryo predicted to be euploid is selected for embryo transfer during in vitro fertilization, and/or wherein an embryo predicted to be aneuploid is not selected for embryo transfer during in vitro fertilization.

48. The method of claim 1, wherein the method is automated.

49.-51. (canceled)
